SEQUENCE LISTING (2 sequences)

SEQ ID NO: 1 - DNA, 50 bp, Artificial Sequence
  16S_V3_F
  misc_feature (42)..(42): any nucleotide
tcgtcggcagcgtcagatgtgtataagagacagcctacgggnggcwgcag

SEQ ID NO: 2 - DNA, 55 bp, Artificial Sequence
  16S_V4_R
gtctcgtgggctcggagatgtgtataagagacaggactachvgggtatctaatcc

(19) United States
(12) Patent Application Publication
(10) Pub. No.: US 2020/0199654 A1
(43) Pub. Date: Jun. 25, 2020

(54) METHOD FOR DIAGNOSING ATOPIC DERMATITIS THROUGH MICROBIAL METAGENOMIC ANALYSIS

(71) Applicant: MD HEALTHCARE INC., Seoul (KR)

(72) Inventor: Yoon-Keun Kim, Gyeonggi-do (KR)

(21) Appl. No.: 16/619,999

(22) PCT Filed: Apr. 26, 2018
(86) PCT No.: PCT/KR2018/004858

§ 371 Date: Dec. 6, 2019

(30) Foreign Application Priority Data
Jun. 7, 2017 (KR) 10-2017-0070879
Apr. 25, 2018 (KR) 10-2018-0048161

(51) Int. Cl. C12Q 1/689

(52) U.S. Cl. CPC C12Q 1/689

(57) ABSTRACT

The present invention relates to a method of diagnosing atopic dermatitis through microbial metagenomic analysis, and more specifically, the present invention relates to a method of diagnosing atopic dermatitis by analyzing an increase or decrease in the content of specific bacteria- or archaea-derived extracellular vesicles by conducting a metagenome analysis using normal individual and subject-derived samples. Since the extracellular vesicles secreted from microorganisms present in the environment can be absorbed into the human body to regulate immune functions and directly affect the occurrence of inflammation. It is difficult to early diagnose atopic dermatitis before symptoms appear, and thus it is difficult to effectively treat the atopic dermatitis. Therefore, as the present invention can early diagnose and predict a risk group of atopic dermatitis by predicting the risk of developing atopic dermatitis through the metagenome analysis using human body-derived samples, it is possible to delay the onset time or prevent the onset of atopic dermatitis through proper management. In addition, the present invention enables early diagnosis even after onset, thereby lowering the incidence of atopic dermatitis and improving therapeutic effects.

### TECHNICAL FIELD

The present invention relates to a method of diagnosing atopic dermatitis through microbial metagenomic analysis, and more particularly, to a method of diagnosing atopic dermatitis by analyzing an increase or decrease in the content of specific bacteria- and archaea-derived extracellular vesicles through microbial metagenomic analysis of bacteria or archaea using normal individual and subject-derived samples.

### BACKGROUND ART

Atopy means having a congenitally irritable allergic property, and in addition to atopy, a chronic skin disease with “inflammation” is called atopic dermatitis. Sometimes, the “atopic dermatitis” is shortened to “atopy”. Atopy is often seen in children and improves as the children become adults, but sometimes it progresses to adults as well. According to a cohort study, atopy increased from 5.1% in 1946 to 7.3% in 1958 and to 12.2% in 1970 in the UK, and increased from 7.05% in 1979 to 18.28% in 1991 in Sweden, and increased from 15% in 1985 to 22.9% in 1997 in Osaka. Japan. In Korea, the incidence of atopic dermatitis in the 2000s was 24.9% for elementary school students and 12.8% for middle school students.

Atopic dermatitis is a chronic inflammatory disease generated by a combination of various factors, and a skin barrier function plays a pivotal role in pathophysiology. As causative factors, foods such as milk are important before the age of 1, and after the age 1, inhaling allergens such as a house dust mite allergen, is known to be important, and recently, bacteria symbiotic on the skin, especially, Staphylococcus aureus, have been known to be important factors. In addition, atopic dermatitis also takes a serious turn due to stress.

Meanwhile, it is known that the number of microorganisms symbiotically living in the human body is 100 trillion which is 10 times the number of human cells, and the number of genes of microorganisms exceeds 100 times the number of human genes. A microbiota or microbiome is a microbial community that includes bacteria, archaea, and eukaryotes present in a given habitat. The intestinal microbiota is known to play a vital role in human's physiological phenomena and significantly affect human health and diseases through interactions with human cells. Bacteria coexisting in human bodies secrete nanometer-sized vesicles to exchange information about genes, proteins, and the like with other cells. The mucous membranes form a physical barrier membrane that does not allow particles with the size of 200 nm or more to pass therethrough, and thus bacteria symbiotically living in the mucous membranes are unable to pass therethrough but bacteria-derived extracellular vesicles have a size of approximately 100 nm or less and thus relatively freely pass through the mucous membranes and are absorbed into the human body.

Metagenomics, also called environmental genomics, may be analytics for metagenomic data obtained from samples collected from the environment (Korean Patent Publication No. 2011-073049). Recently, the bacterial composition of human microbiota has been listed using a method based on 16s ribosomal RNA (16s rRNA) base sequences, and 16s rDNA base sequences, which are genes of 16s ribosomal RNA, are analyzed using a next generation sequencing (NGS) platform. However, in the onset of atopic dermatitis, identification of causative factors of atopic dermatitis through metagenomic analysis of microorganisms-derived vesicles isolated from a human-derived substance, such as blood or urine and the like, and a method of predicting atopic dermatitis have never been reported.

### DISCLOSURE

### Technical Problem

The inventors isolated extracellular vesicles from normal individual and subject-derived samples such as blood and urine, extracted genes from the vesicles, and conducted metagenomic analysis thereof to diagnose atopic dermatitis. As a result, bacteria- and archaea-derived extracellular vesicles, which can serve as causative factors of atopic dermatitis were identified, and based on this, the present invention was completed.

Therefore, the present invention was directed to providing a method of providing information to diagnose atopic dermatitis through metagenomic analysis of bacteria- and archaea-derived extracellular vesicles, a method of diagnosing atopic dermatitis, and a method of predicting the risk of the onset of atopic dermatitis.

However, the technical goals of the present invention are not limited to the aforementioned goals,and other unmentioned technical goals will be clearly understood by those of ordinary skill in the art from the following description.

### Technical Solution

To achieve the above-described object of the present invention, there is provided a method of providing information for atopic dermatitis diagnosis, comprising the following processes:

(a) extracting DNAs from extracellular vesicles isolated from normal individual and subject samples;

(b) performing polymerase chain reaction (PCR) on the extracted DNA using a pair of primers comprising SEQ ID NO: 1 and SEQ ID NO: 2; and

(c) comparing an increase or decrease in content of bacteria- and archaea-derived extracellular vesicles of the subject-derived sample with that of a normal individual-derived sample through sequencing of a product of the PCR.

The present invention also provides a method of diagnosing atopic dermatitis, comprising the following processes:

(a) extracting DNAs from extracellular vesicles isolated from normal individual and subject samples;

(b) performing polymerase chain reaction (PCR) on the extracted DNA using a pair of primers comprising SEQ ID NO: 1 and SEQ ID NO: 2; and

(c) comparing an increase or decrease in content of bacteria- and archaea-derived extracellular vesicles of the subject-derived sample with that of a normal individual-derived sample through sequencing of a product of the PCR.

The present invention also provides a method of predicting a risk for atopic dermatitis, comprising the following processes:

(a) extracting DNAs from extracellular vesicles isolated from normal individual and subject samples;

(b) performing polymerase chain reaction (PCR) on the extracted DNA using a pair of primers comprising SEQ ID NO: 1 and SEQ ID NO: 2; and

(c) comparing an increase or decrease in content of bacteria- and archaea-derived extracellular vesicles of the subject-derived sample with that of a normal individual-derived sample through sequencing of a product of the PCR.

In one embodiment of the present invention, the normal individual and subject samples may be blood or urine.

In another exemplary embodiment of the present invention, in the step (c), an increase or decrease in content of extracellular vesicles derived from one or more bacteria selected from the group consisting of the phylum Cyanobacteria, the phylum Fusobacteria, the phylum Verrucomicrobia, the phylum Euryarchaeota, the phylum Firmicutes, the phylum Bacteroidetes and the phylum Tenericutes may be compared.

In another exemplary embodiment of the present invention, in the step (c), an increase or decrease in content of extracellular vesicles derived from one or more bacteria selected from the group consisting of the class Chloroplast, the class Saprospirae, the class Flavobacteriia, the class Alphaproteobacteria, the class Fusobacteriia, the class Bacilli, the class Verrucomicrobiae, the class Methanobacteria, the class Betaproteobacteria, the class Coriobacteriia, the class Clostridia, the class Bacteroidia, the class Erysipelotrichi, the class Mollicutes, and the class Pedosphaerae may be compared.

In another exemplary embodiment of the present invention, in the step (c), an increase or decrease in content of extracellular vesicles derived from one or more bacteria selected from the group consisting of the order Stramenopiles, the order Pseudomonadales, the order Neisseriales, the order Streptophyta, the order Rhizobiales, the order Saprospirales, the order Sphingomonadales, the order Flavobacteriales, the order Caulobacterales, the order Gemellales, the order Pasteurellales, the order Fusobacteriales, the order Rhodobacterales, the order Bacillales, the order Oceanospirillales, the order Enterobacteriales, the order Bifidobacteriales, the order Verrucomicrobiales, the order Methanobacteriales, the order Desulfovibrionales, the order MLE1-12, the order Burkholderiales, the order Coriobacteriales, the order Clostridiales, the order Bacteroidales, the order Erysipelotrichales, the order Turicibacterales, the order RF39, and the order Pedosphaerales may be compared.

In another exemplary embodiment of the present invention, in the step (c), an increase or decrease in content of extracellular vesicles derived from one or more bacteria selected from the group consisting of the family Exiguobacteraceae, the family Moraxellaceae, the family Bradyrhizobiaceae, the family Rhizobiaceae, the family Flavobacteriaceae, the family Campylobacteraceae, the family Neisseriaceae, the family Pseudomonadaceae, the family Sphingomonadacecae, the family Chitinophagaceae, the family Carnobacteriaceae, the family Caulobacteraceae, the family Weeksellaceae, the family Methylobacteriaceae, the family Gemellaceae, the family Dermabacteraceae, the family Propionibacteriaceae, the family Pasteurellaceae, the family Leptotrichiaceae, the family Oxalobacteraceae, the family Fusobacteriaceae, the family Aerococcaceae, the family Rhodobacteraceae, the family Intrasporangiaceae, the family Paraprevotellaceae, the family Porphyromonadaceae, the family Staphylococcaceae, the family Corynebacteriaceae, the family Tissierellaceae, the family Micrococcaceae, the family Actinomycetaceae, the family Planococcaceae, the family Alcaligenaceae, the family mitochondria, the family Comamonadaceae, the family Veillonellaceae, the family Bifidobacteriaceae, the family Coriobacteriaceae, the family Clostridiaceae, the family Erysipelotrichaceae, the family Turicibacteraceae, the family Lachnospiraceae, the family Prevotellaceae, the family Rikenellaceae, the family Bacteroidaceae, the family Enterococcaceae, the family Ruminococcaceae, the family Desulfovibrionaceae, the family Verrucomicrobiaceae, the family Odoribacteraceae, the family Christensenellaceae, the family Methanobacteriaceae, the family Koribacteraceae, and the family Streptomycetaceae may be compared.

In another exemplary embodiment of the present invention, in the step (c), an increase or decrease in content of extracellular vesicles derived from one or more bacteria selected from the group consisting of the genus Exiguobacterium, the genus Acinetobacter, the genus Capnocytophaga, the genus Proteus, the genus Neisseria, the genus Sphingomonas, the genus Pseudomonas, the genus Aggregatibacter, the genus Leptotrichia, the genus Granulicatella, the genus Prevotella, the genus Chryseobacterium, the genus Porphyromonas, the genus Haemophilus, the genus Brachybacterium, the genus Propionibacterium, the genus Eubacterium, the genus Fusobacterium, the genus Enhydrobacter, the genus Paracoccus, the genus Parabacteroides, the genus Staphylococcus, the genus Corynebacterium, the genus Rothia, the genus Actinomyces, the genus Dialister, the genus Faecalibacterium, the genus Dorea, the genus Ruminococcus, the genus Halomonas, the genus Sutterella, the genus Bacteroides, the genus Veillonella, the genus Rhodococcus, the genus Butyricimonas, the genus Akkermansia, the genus Bifidobacterium, the genus Atopobium, the genus Citrobacter, the genus Klebsiella, the genus Enterobacter, the genus Chromohalobacter, the genus Cupriavidus, the genus Methanobrevibacter, the genus Phascolarctobacterium, the genus Odoribacter, the genus Pyramidobacter, the genus Bilophila, the genus Desulfovibrio, the genus Acidaminococcus, the genus Achromobacter, the genus Agrobacterium, the genus Roseateles, the genus Clostridium, the genus Coprococcus, the genus Turicibacter, the genus Roseburia, the genus Lachnospira, the genus Blautia, the genus Oscillospira, the genus Enterococcus, the genus SMB53, the genus Catenibacterium, the genus Paraprevotella, the genus Adlercreutzia, the genus Slackia, and the genus Thermoanaerobacterium may be compared.

In still another exemplary embodiment of the present invention, in the step (c), an increase or decrease in content of extracellular vesicles derived from: one or more bacteria selected from the group consisting of the phylum Cyanobacteria, the phylum Fusobacteria, the phylum Verrucomicrobia and the phylum Euryarchaeota, which are isolated from normal individual and subject-derived blood samples, and the phylum Cyanobacteria, the phylum Firmicutes, the phylum Bacteroidetes, the phylum Verrucomicrobia, the phylum Euryarchaeota and the phylum Tenericutes, which are isolated from normal individual and subject-derived urine samples;

one or more bacteria selected from the group consisting of the class Chloroplast, the class Saprospirae, the class Flavobacteriia, the class Alphaproteobacteria, the class Fusobacteriia, the class Bacilli, the class Verrucomicrobiae and the class Methanobacteria, which are isolated from normal individual and subject-derived blood samples, and the class Chloroplast, the class Betaproteobacteria, the class Coriobacteriia, the class Clostridia, the class Bacteroidia, the class Erysipelotrichi, the class Verrucomicrobiae, the class Methanobacteria, the class Mollicutes and the class Pedosphaerae, which are isolated from normal individual and subject-derived urine samples;

one or more bacteria selected from the group consisting of the order Stramenopiles, the order Pseudomonadales, the order Neisseriales, the order Streptophyta, the order Rhizobiales, the order Saprospirales, the order Sphingomonadales, the order Flavobacteriales, the order Caulobacterales, the order Gemellales, the order Pasteurellales, the order Fusobacteriales, the order Rhodobacterales, the order Bacillales, the order Lactobacillales, the order Oceanospirillales, the order Enterobacteriales, the order Bifidobacteriales, the order Verrucomicrobiales, the order Methanobacteriales and the order Desulfovibrionales, which are isolated from normal individual and subject-derived blood samples, and MLE1-12, the order Burkholderiales, the order Streptophyta, the order Pseudomonadales, the order Sphingomonadales, the order Bifidobacteriales, the order Coriobacteriales, the order Clostridiales, the order Bacteroidales, the order Erysipelotrichales, the order Turicibacterales, the order Desulfovibrionales, the order Verrucomicrobiales, the order Methanobacteriales, the order RF39 and the order Pedosphaerales, which are isolated from normal individual and subject-derived urine samples;

one or more bacteria selected from the group consisting of the family Exiguobacteraceae, the family Moraxellaceae, the family Bradyrhizobiaceae, the family Rhizobiaceae, the family Flavobacteriaceae, the family Campylobacteraceae, the family Neisseriaceae, the family Pseudomonadaceae, the family Sphingomonadaceae, the family Chitinophagaceae, the family Carnobacteriaceae, the family Caulobacteraceae, the family Weeksellaceae, the family Methylobacteriaceae, the family Gemellaceae, the family Dermabacteraceae, the family Propionibacteriaceae, the family Pasteurellaceae, the family Leptotrichiaceae, the family Oxalobacteraceae, the family Fusobacteriaceae, the family Aerococcaceae, the family Rhodobacteraceae, the family Intrasporangiaceae, the family Paraprevotellaceae, the family Porphyromonadaceae, the family Staphylococcaceae, the family Corynebacteriaceae, the family Tissierellaceae, the family Micrococcaceae, the family Actinomycetaceae, the family Planococcaceae, the family Comamonadaceae, the family Halomonadaceae, the family Clostridiaceae, the family Alcaligenaceae, the family Enterobacteriaceae, the family Bacteroidaceae, the family Peptostreptococcaceae, the family Nocardiaceae, the family Bifidobacteriaceae, the family Verrucomicrobiaceae, the family Shewanellaceae, the family Barnesiellaceae, the family Odoribacteraceae, the family Methanobacteriaceae, the family Rikenellaceae, the family Desulfovibrionaceae, and the family Dethiosulfovibrionaceae, which are isolated from normal individual and subject-derived blood samples, and the family Alcaligenaceae, the family Rhizobiaceae, the family mitochondria, the family Pseudomonadaceae, the family Corynebacteriaceae, the family Comamonadaceae, the family Rhodobacteraceae, the family Sphingomonadaceae, the family Veillonellaceae, the family Bifidobacteriaceae, the family Coriobacteriaceae, the family Planococcaceae, the family Paraprevotellaceae, the family Clostridiaceae, the family Erysipelotrichaceae, the family Turicibacteraceae, the family Lachnospiraceae, the family Prevotellaceae, the family Rikenellaceae, the family Bacteroidaceae, the family Enterococcaceae, the family Ruminococcaceae, the family Desulfovibrionaceae, the family Verrucomicrobiaceae, the family Odoribacteraceae, the family Christensenellaceae, the family Methanobacteriaceae, the family Koribacteraceae and the family Streptomycetaceae, which are isolated from normal individual and subject-derived urine samples; or

one or more bacteria selected from the group consisting of the genus Exiguobacterium, the genus Acinetobacter, the genus Capnocytophaga, the genus Proteus, the genus Neisseria, the genus Sphingomonas, the genus Pseudomonas, the genus Aggregatibacter, the genus Leptotrichia, the genus Granulicatella, the genus Prevotella, the genus Chryseobacterium, the genus Porphyromonas, the genus Haemophilus, the genus Brachybacterium, the genus Propionibacterium, the genus Eubacterium, the genus Fusobacterium, the genus Enhydrobacter, the genus Paracoccus, the genus Parabacteroides, the genus Staphylococcus, the genus Corynebacterium, the genus Rothia, the genus Actinomyces, the genus Dialister, the genus Faecalibacterium, the genus Dorea, the genus Ruminococcus, the genus Halomonas, the genus Sutterella, the genus Bacteroides, the genus Veillonella, the genus Rhodococcus, the genus Butyricimonas, the genus Akkermansia, the genus Bifidobacterium, the genus Atopobium, the genus Citrobacter, the genus Klebsiella, the genus Enterobacter, the genus Chromohalobacter, the genus Cupriavidus, the genus Methanobrevibacter, the genus Phascolarctobacterium, the genus Odoribacter, the genus Pyramidobacter, the genus Bilophila, the genus Desulfovibrio and the genus Acidaminococcus, which are isolated from normal individual and subject-derived blood samples, and the genus Achromobacter, the genus Agrobacterium, the genus Roseateles, the genus Pseudomonas, the genus Corynebacterium, the genus Sphingomonas, the genus Citrobacter, the genus Faecalibacterium, the genus Clostridium, the genus Coprococcus, the genus Dialister, the genus Bifidobacterium, the genus Turicibacter, the genus Dorea, the genus Sutterella, the genus Ruminococcus, the genus Prevotella, the genus Roseburia, the genus Bacteroides, the genus Klebsiella, the genus Lachnospira, the genus Blautia, the genus Cupriavidus, the genus Oscillospira, the genus Enterococcus, the genus SMB53, the genus Akkermansia, the genus Parabacteroides, the genus Phascolarctobacterium, the genus Catenibacterium, the genus Butyricimonas, the genus Eubacterium, the genus Halomonas, the genus Paraprevotella, the genus Methanobrevibacter, the genus Adlercreutzia, the genus Slackia, the genus Desulfovibrio and the genus Thermoanaerobacterium, which are isolated from normal individual and subject-derived urine samples, may be compared.

In still another exemplary embodiment of the present invention, in the step (c), in comparison with the normal individual-derived sample, it is possible to diagnose an increase in the content of the following as atopic dermatitis:

extracellular vesicles derived from one or more bacteria selected from the group consisting of the phylum Verrucomicrobia, and the phylum Euryarchaeota, which are isolated from subject-derived blood samples, and the phylum Firmicutes, the phylum Bacteroidetes, the phylum Verrucomicrobia, the phylum Euryarchaeota, and the phylum Tenericutes, which are isolated from subject-derived urine samples,

extracellular vesicles derived from one or more bacteria selected from the group consisting of the class Bacilli, the class Verrucomicrobiae, and the class Methanobacteria, which are isolated from subject-derived blood samples, and the class Coriobacteriia, the class Clostridia, the class Bacteroidia, the class Erysipelotrichi, the class Verrucomicrobiae, the class Methanobacteria, the class Mollicutes, and the class Pedosphaerae, which are isolated from subject-derived urine samples,

extracellular vesicles derived from one or more bacteria selected from the group consisting of the order Lactobacillales, the order Oceanospirillales, the order Enterobacteriales, the order Bifidobacteriales, the order Verrucomicrobiales, the order Methanobacteriales, and the order Desulfovibrionales, which are isolated from subject-derived blood samples, and the order Bifidobacteriales, the order Coriobacteriales, the order Clostridiales, the order Bacteroidales, the order Erysipelotrichales, the order Turicibacterales, the order Desulfovibrionales, the order Verrucomicrobiales, the order Methanobacteriales, the order RF39, and the order Pedosphaerales, which are isolated from subject-derived urine samples,

extracellular vesicles derived from one or more bacteria selected from the group consisting of the family Comamonadaceae, the family Halomonadaceae, the family Clostridiaceae, the family Alcaligenaceae, the family Enterobacteriaceae, the family Bacteroidaceae, the family Peptostreptococcaceae, the family Nocardiaceae, the family Bifidobacteriaceae, the family Verrucomicrobiaceae, the family Shewanellaceae, the family Barnesiellaceae, the family Odoribacteraceae, the family Methanobacteriaceae, the family Rikenellaceae, the family Desulfovibrionaceae, and the family Dethiosulfovibrionaceae, which are isolated from subject-derived blood samples, and the family Veillonellaceae, the family Bifidobacteriaceae, the family Coriobacteriaceae, the family Planococcaceae, the family Paraprevotellaceae, the family Clostridiaceae, the family Erysipelotrichaceae, the family Turicibacteraceae, the family Lachnospiraceae, the family Prevotellaceae, the family Rikenellaceae, the family Bacteroidaceae, the family Enterococcaceae, the family Ruminococcaceae, the family Desulfovibrionaceae, the family Verrucomicrobiaceae, the family Odoribacteraceae, the family Christensenellaceae, the family Methanobacteriaceae, the family Koribacteraceae, and the family Streptomycetaceae, which are isolated from subject-derived urine samples, or

extracellular vesicles derived from one or more bacteria selected from the group consisting of the genus Ruminococcus, the genus Halomonas, the genus Sutterella, the genus Bacteroides, the genus Veillonella, the genus Rhodococcus, the genus Butyricimonas, the genus Akkermansia, the genus Bifidobacterium, the genus Atopobium, the genus Citrobacter, the genus Klebsiella, the genus Enterobacter, the genus Chromohalobacter, the genus Cupriavidus, the genus Methanobrevibacter, the genus Phascolarctobacterium, the genus Odoribacter, the genus Pyramidobacter, the genus Bilophila, the genus Desulfovibrio, and the genus Acidaminococcus, which are isolated from subject-derived blood samples, and the genus Citrobacter, the genus Faecalibacterium, the genus Clostridium, the genus Coprococcus, the genus Dialister, the genus Bifidobacterium, the genus Turicibacter, the genus Dorea, the genus Sutterella, the genus Ruminococcus, the genus Prevotella, the genus Roseburia, the genus Bacteroides, the genus Klebsiella, the genus Lachnospira, the genus Blautia, the genus Cupriavidus, the genus Oscillospira, the genus Enterococcus, the genus Ruminococcus, the genus SMB53, the genus Akkermansia, the genus Parabacteroides, the genus Phascolarctobacterium, the genus Catenibacterium, the genus Butyricimonas, the genus Eubacterium, the genus Halomonas, the genus Paraprevotella, the genus Methanobrevibacter, the genus Adlercreutzia, the genus Slackia, the genus Desulfovibrio, and the genus Thermoanaerobacterium, which are isolated from subject-derived urine samples.

In still another exemplary embodiment of the present invention, in the step (c), in comparison with the normal individual-derived sample, it is possible to diagnose a decrease in the content of the following as atopic dermatitis:

extracellular vesicles derived from one or more bacteria selected from the group consisting of the phylum Cyanobacteria, and the phylum Fusobacteria, which are isolated from subject-derived blood samples, and the phylum Cyanobacteria, which are isolated from subject-derived urine samples,

extracellular vesicles derived from one or more bacteria selected from the group consisting of the class Chloroplast, the class Saprospirae, the class Flavobacteriia, the class Alphaproteobacteria, and the class Fusobacteriia, which are isolated from subject-derived blood samples, and the class Chloroplast, and the class Betaproteobacteria, which are isolated from subject-derived urine samples,

extracellular vesicles derived from one or more bacteria selected from the group consisting of the order Stramenopiles, the order Pseudomonadales, the order Neisseriales, the order Streptophyta, the order Rhizobiales, the order Saprospirales, the order Sphingomonadales, the order Flavobacteriales, the order Caulobacterales, the order Gemellales, the order Pasteurellales, the order Fusobacteriales, the order Rhodobacterales, and the order Bacillales, which are isolated from subject-derived blood samples, and the order MLE1-12, the order Burkholderiales, the order Streptophyta, the order Pseudomonadales, and the order Sphingomonadales, which are isolated from subject-derived urine samples,

extracellular vesicles derived from one or more bacteria selected from the group consisting of the family Exiguobacteraceae, the family Moraxellaceae, the family Bradyrhizobiaceae, the family Rhizobiaceae, the family Flavobacteriaceae, the family Campylobacteraceae, the family Neisseriaceae, the family Pseudomonadaceae, the family Sphingomonadaceae, the family Chitinophagaceae, the family Carnobacteriaceae, the family Caulobacteraceae, the family Weeksellaceae, the family Methylobacteriaceae, the family Gemellaceae, the family Dermabacteraceae, the family Propionibacteriaceae, the family Pasteurellaceae, the family Leptotrichiaceae, the family Oxalobacteraceae, the family Fusobacteriaceae, the family Aerococcaceae, the family Rhodobacteracecae, the family Intrasporangiaceae, the family Paraprevotellaceae, the family Porphyromonadaceae, the family Staphylococcaceae, the family Corynebacteriaceae, the family Tissierellaceae, the family Micrococcaceae, the family Actinomycetaceae, and the family Planococcaceae, which are isolated from subject-derived blood samples, and the family Alcaligenaceae, the family Rhizobiaceae, the family mitochondria, the family Pseudomonadaceae, the family Corynebacteriaceae, the family Comamonadaceae, the family Rhodobacteraceae, and the family Sphingomonadaceae, which are isolated from subject-derived urine samples, or

extracellular vesicles derived from one or more bacteria selected from the group consisting of the genus Exiguobacterium, the genus Acinetobacter, the genus Capnocytophaga, the genus Proteus, the genus Neisseria, the genus Sphingomonas, the genus Pseudomonas, the genus Aggregatibacter, the genus Leptotrichia, the genus Granulicatella, the genus Prevotella, the genus Chryseobacterium, the genus Porphyromonas, the genus Haemophilus, the genus Brachybacterium, the genus Propionibacterium, the genus Eubacterium, the genus Fusobacterium, the genus Enhydrobacter, the genus Paracoccus, the genus Parabacteroides, the genus Staphylococcus, the genus Corynebacterium, the genus Rothia, the genus Actinomyces, the genus Dialister, the genus Faecalibacterium, and the genus Dorea, which are isolated from subject-derived blood samples, and the genus Achromobacter, the genus Agrobacterium, the genus Roseateles, the genus Pseudomonas, the genus Corynebacterium, and the genus Sphingomonas, which are isolated from subject-derived urine samples.

In still another exemplary embodiment of the present invention, the blood may be whole blood, serum, plasma, or blood mononuclear cells.

### Advantageous Effects

Extracellular vesicles secreted from microorganisms in the environment can be absorbed into the body and directly affect immune function regulation and inflammation, and atopic dermatitis is difficult to be efficiently treated because early diagnosis is difficult before symptoms appear. Therefore, by predicting the risk of atopic dermatitis onset in advance through the metagenomic analysis of bacteria-derived extracellular vesicles using human body-derived samples according to the present invention, it is possible to diagnose and predict an atopic dermatitis risk group early, and to delay the onset time or prevent the onset of atopic dermatitis through proper management. In addition, even after the onset, since it is possible to diagnose atopic dermatitis early, there are advantages in that it is possible to lower the incidence of atopic dermatitis and increase therapeutic effects, and it is possible to ameliorate the progression of the disease or prevent recurrence by avoiding exposure to causal factors through a metagenomic analysis in a patient diagnosed with atopic dermatitis.

### DESCRIPTION OF DRAWINGS

FIG. 1A illustrates images showing the distribution pattern of bacteria and extracellular vesicles over time after intestinal bacteria and bacteria-derived extracellular vesicles (EVs) were orally administered to mice, and FIG. 1B illustrates images showing the distribution pattern of bacteria and EVs after being orally administered to mice and, at 12 hours, blood and various organs were extracted.

FIG. 2 shows the distribution of vesicles (extracellular vesicles; EVs) derived from bacteria, which is significant in diagnostic performance at a phylum level by isolating bacteria-derived vesicles from blood of a patient with atopic dermatitis and normal individual, and then performing metagenomic analysis.

FIG. 3 shows the distribution of vesicles (extracellular vesicles; EVs) derived from bacteria, which is significant in diagnostic performance at a class level by isolating bacteria-derived vesicles from blood of a patient with atopic dermatitis and normal individual, and then performing metagenomic analysis.

FIGS. 4A and 4B show the distribution of vesicles (extracellular vesicles; EVs) derived from bacteria, which is significant in diagnostic performance at an order level by isolating bacteria-derived vesicles from blood of a patient with atopic dermatitis and normal individual, and then performing metagenomic analysis.

FIGS. 5A and 5B show the distribution of vesicles (extracellular vesicles; EVs) derived from bacteria, which is significant in diagnostic performance at a family level by isolating bacteria-derived vesicles from blood of a patient with atopic dermatitis and normal individual, and then performing metagenomic analysis.

FIGS. 6A and 6B show the distribution of vesicles (extracellular vesicles; EVs) derived from bacteria, which is significant in diagnostic performance at a genus level by isolating bacteria-derived vesicles from blood of a patient with atopic dermatitis and normal individual, and then performing metagenomic analysis.

FIG. 7 shows the distribution of vesicles extracellular vesicles; EVs) derived from bacteria, which is significant in diagnostic performance at a phylum level by isolating bacteria-derived vesicles from urine of a patient with atopic dermatitis and normal individual, and then performing metagenomic analysis.

FIG. 8 shows the distribution of vesicles (extracellular vesicles; EVs) derived from bacteria, which is significant in diagnostic performance at a class level by isolating bacteria-derived vesicles from urine of a patient with atopic dermatitis and normal individual, and then performing metagenomic analysis.

FIG. 9 shows the distribution of vesicles (extracellular vesicles; EVs) derived from bacteria, which is significant in diagnostic performance at an order level by isolating bacteria-derived vesicles from urine of a patient with atopic dermatitis and normal individual, and then performing metagenomic analysis.

FIGS. 10A and 10B show the distribution of vesicles (extracellular vesicles; EVs) derived from bacteria, which is significant in diagnostic performance at a family level by isolating bacteria-derived vesicles from urine of a patient with atopic dermatitis and normal individual, and then performing metagenomic analysis.

FIGS. 11A and 11B show the distribution of vesicles (extracellular vesicles; EVs) derived from bacteria, which is significant in diagnostic performance at a genus level by isolating bacteria-derived vesicles from urine of a patient with atopic dermatitis and normal individual, and then performing metagenomic analysis.

### BEST MODE

The present invention relates to a method of diagnosing atopic dermatitis through microorganisms metagenomic analysis. The inventors of the present invention extracted genes from extracellular vesicles using a normal individual and a subject-derived sample, performed metagenomic analysis thereon, and identified bacteria-derived extracellular vesicles capable of acting as a causative factor of atopic dermatitis.

Therefore, the present invention provides a method of providing information for diagnosing atopic dermatitis, the method comprising:

(a) extracting DNAs from extracellular vesicles isolated from normal individual and subject samples;

(b) performing polymerase chain reaction (PCR) on the extracted DNA using a pair of primers comprising SEQ ID NO: 1 and SEQ ID NO: 2; and

(c) comparing an increase or decrease in content of bacteria- and archaea-derived extracellular vesicles of the subject-derived sample with that of a normal individual-derived sample through sequencing of a product of the PCR.

The term “atopic dermatitis diagnosis” as used herein refers to determining whether a patient has a risk for atopic dermatitis, whether the risk for atopic dermatitis is relatively high, or whether atopic dermatitis has already occurred. The method of the present invention may be used to delay the onset of atopic dermatitis through special and appropriate care for a specific patient, which is a patient having a high risk for atopic dermatitis or prevent the onset of atopic dermatitis. In addition, the method may be clinically used to determine treatment by selecting the most appropriate treatment method through early diagnosis of atopic dermatitis.

The term “metagenome” as used herein refers to the total of genomes including all viruses, bacteria, fungi, and the like in isolated regions such as soil, the intestines of animals, and the like, and is mainly used as a concept of genomes that explains identification of many microorganisms at once using a sequencer to analyze non-cultured microorganisms. In particular, a metagenome does not refer to a genome of one species, but refers to a mixture of genomes, including genomes of all species of an environmental unit. This term originates from the view that, when defining one species in a process in which biology is advanced into omics, various species as well as existing one species functionally interact with each other to form a complete species. Technically, it is the subject of techniques that analyzes all DNAs and RNAs regardless of species using rapid sequencing to identify all species in one environment and verify interactions and metabolism. In the present invention, bacterial metagenomic analysis is performed using bacteria-derived extracellular vesicles isolated from, for example, blood and urine.

The term “bacteria-derived vesicles” used herein is the generic term for extracellular vesicles secreted from archaea as well as bacteria, but the present invention is not limited thereto.

In the present invention, the normal individual and subject samples may be blood or urine, and the blood is preferably whole blood, serum, plasma or blood monocytes, but the present invention is not limited thereto.

In an embodiment of the present invention, metagenomic analysis is performed on the bacteria- and archaea-derived extracellular vesicles, and bacteria-derived extracellular vesicles capable of acting as a cause of the onset of atopic dermatitis were actually identified by analysis at phylum, class, order, family, and genus levels.

More particularly, in one embodiment of the present invention, as a result of performing bacterial metagenomic analysis on extracellular vesicles present in subject-derived blood samples at a phylum level, the content of extracellular vesicles derived from bacteria belonging to the phylum Cyanobacteria, the phylum Fusobacteria, the phylum Verrucomicrobia, and the phylum Euryarchaeota was significantly different between atopic dermatitis patients and normal individuals (see Example 4).

More particularly, in one embodiment of the present invention, as a result of performing bacterial metagenomic analysis on extracellular vesicles present in subject-derived blood samples at a class level, the content of extracellular vesicles derived from bacteria belonging to the class Chloroplast, the class Saprospirae, the class Flavobacteriia, the class Alphaproteobacteria, the class Fusobacteriia, the class Bacilli, the class Verrucomicrobiae, and the class Methanobacteria was significantly different between atopic dermatitis patients and normal individuals (see Example 4).

More particularly, in one embodiment of the present invention, as a result of performing bacterial metagenomic analysis on extracellular vesicles present in subject-derived blood samples at an order level, the content of extracellular vesicles derived from bacteria belonging to the order Stramenopiles, the order Pseudomonadales, the order Neisseriales, the order Streptophyta, the order Rhizobiales, the order Saprospirales, the order Sphingomonadales, the order Flavobacteriales, the order Caulobacterales, the order Gemellales, the order Pasteurellales, the order Fusobacteriales, the order Rhodobacterales, the order Bacillales, the order Lactobacillales, the order Oceanospirillales, the order Enterobacteriales, the order Bifidobacteriales, the order Verrucomicrobiales, the order Methanobacteriales, and the order Desulfovibrionales was significantly different between atopic dermatitis patients and normal individuals (see Example 4).

More particularly, in one embodiment of the present invention, as a result of performing bacterial metagenomic analysis on extracellular vesicles present in subject-derived blood samples at a family level, the content of extracellular vesicles derived from bacteria belonging to the family Exiguobacteraceae, the family Moraxellaceae, the family Bradyrhizobiaceae, the family Rhizobiaceae, the family Flavobacteriaceae, the family Campylobacteraceae, the family Neisseriaceae, the family Pseudomonadaceae, the family Sphingomonadaceae, the family Chitinophagaceae, the family Carnobacteriaceae, the family Caulobacteraceae, the family Weeksellaceae, the family Methylobacteriaceae, the family Gemellaceae, the family Dermabacteraceae, the family Propionibacteriaceae, the family Pasteurellaceae, the family Leptotrichiaceae, the family Oxalobacteraceae, the family Fusobacteriaceae, the family Aerococcaceae, the family Rhodobacteraceae, the family Intrasporangiaceae, the family Paraprevotellaceae, the family Porphyromonadaceae, the family Staphylococcaceae, the family Corynebacteriaceae, the family Tissierellaceae, the family Micrococcaceae, the family Actinomycetaceae, the family Planococcaceae, the family Comamonadaceae, the family Halomonadaceae, the family Clostridiaceae, the family Alcaligenaceae, the family Enterobacteriaceae, the family Bacteroidaceae, the family Peptostreptococcaceae, the family Nocardiaceae, the family Bifidobacteriaceae, the family Verrucomicrobiaceae, the family Shewanellaceae, the family Barnesiellaceae, the family Odoribacteraceae, the family Methanobacteriaceae, the family Rikenellaceae, the family Desulfovibrionaceae, and the family Dethiosulfovibrionaceae was significantly different between atopic dermatitis patients and normal individuals (see Example 4).

More particularly, in one embodiment of the present invention, as a result of performing bacterial metagenomic analysis on extracellular vesicles present in subject-derived blood samples at a genus level, the content of extracellular vesicles derived from bacteria belonging to the genus Exiguobacterium, the genus Acinetobacter, the genus Capnocytophaga, the genus Proteus, the genus Neisseria, the genus Sphingomonas, the genus Pseudomonas, the genus Aggregatibacter, the genus Leptotrichia, the genus Granulicatella, the genus Prevotella, the genus Chryseobacterium, the genus Porphyromonas, the genus Haemophilus, the genus Brachybacterium, the genus Propionibacterium, the genus Eubacterium, the genus Fusobacterium, the genus Enhydrobacter, the genus Paracoccus, the genus Parabacteroides, the genus Staphylococcus, the genus Corynebacterium, the genus Rothia, the genus Actinomyces, the genus Dialister, the genus Faecalibacterium, the genus Dorea, the genus Ruminococcus, the genus Halomonas, the genus Sutterella, the genus Bacteroides, the genus Veillonella, the genus Rhodococcus, the genus Butyricimonas, the genus Akkermansia, the genus Bifidobacterium, the genus Atopobium, the genus Citrobacter, the genus Klebsiella, the genus Enterobacter, the genus Chromohalobacter, the genus Cupriavidus, the genus Methanobrevibacter, the genus Phascolarctobacterium, the genus Odoribacter, the genus Pyramidobacter, the genus Bilophila, the genus Desulfovibrio, and the genus Acidaminococcus was significantly different between atopic dermatitis patients and normal individuals (see Example 4).

More particularly, in one embodiment of the present invention, as a result of performing bacterial metagenomic analysis on extracellular vesicles present in subject-derived urine samples at a phylum level, the content of extracellular vesicles derived from bacteria belonging to the phylum Cyanobacteria, the phylum Firmicutes, the phylum Bacteroidetes, the phylum Verrucomicrobia, the phylum Euryarchaeota, and the phylum Tenericutes was significantly different between atopic dermatitis patients and normal individuals (see Example 5).

More particularly, in one embodiment of the present invention, as a result of performing bacterial metagenomic analysis on extracellular vesicles present in subject-derived urine samples at a class level, the content of extracellular vesicles derived from bacteria belonging to the class Chloroplast, the class Betaproteobacteria, the class Coriobacteriia, the class Clostridia, the class Bacteroidia, the class Erysipelotrichi, the class Verrucomicrobiae, the class Methanobacteria, the class Mollicutes, and the class Pedosphaerae was significantly different between atopic dermatitis patients and normal individuals (see Example 5).

More particularly, in one embodiment of the present invention, as a result of performing bacterial metagenomic analysis on extracellular vesicles present in subject-derived urine samples at an order level, the content of extracellular vesicles derived from bacteria belonging to the order MLE1-12, the order Burkholderiales, the order Streptophyta, the order Pseudomonadales, the order Sphingomonadales, the order Bifidobacteriales, the order Coriobacteriales, the order Clostridiales, the order Bacteroidales, the order Erysipelotrichales, the order Turicibacterales, the order Desulfovibrionales, the order Verrucomicrobiales, the order Methanobacteriales, the order RF39, and the order Pedosphaerales was significantly different between atopic dermatitis patients and normal individuals (see Example 5).

More particularly, in one embodiment of the present invention, as a result of performing bacterial metagenomic analysis on extracellular vesicles present in subject-derived urine samples at a family level,the content of extracellular vesicles derived from bacteria belonging to the family Alcaligenaceae, the family Rhizobiaceae, the family mitochondria, the family Pseudomonadaceae, the family Corynebacteriaceae, the family Comamonadaceae, the family Rhodobacteraceae, the family Sphingomonadaceae, the family Veillonellaceae, the family Bifidobacteriaceae, the family Coriobacteriaceae, the family Planococcaceae, the family Paraprevotellaceae, the family Clostridiaceae, the family Erysipelotrichaceae, the family Turicibacteraceae, the family Lachnospiraceae, the family Prevotellaceae, the family Rikenellaceae, the family Bacteroidaceae, the family Enterococcaceae, the family Ruminococcaceae, the family Desulfovibrionaceae, the family Verrucomicrobiaceae, the family Odoribacteraceae, the family Christensenellaceae, the family Methanobacteriaceae, the family Koribacteraceae, and the family Streptomycetaceae was significantly different between atopic dermatitis patients and normal individuals (see Example 5).

More particularly, in one embodiment of the present invention, as a result of performing bacterial metagenomic analysis on extracellular vesicles present in subject-derived urine samples at a genus level, the content of extracellular vesicles derived from bacteria belonging to the genus Achromobacter, the genus Agrobacterium, the genus Roseateles, the genus Pseudomonas, the genus Corynebacterium, the genus Sphingomonas, the genus Citrobacter, the genus Faecalibacterium, the genus Clostridium, the genus Coprococcus, the genus Dialister, the genus Bifidobacterium, the genus Turicibacter, the genus Dorea, the genus Sutterella, the genus Ruminococcus, the genus Prevotella, the genus Roseburia, the genus Bacteroides, the genus Klebsiella, the genus Lachnospira, the genus Blautia, the genus Cupriavidus, the genus Oscillospira, the genus Enterococcus, the genus Ruminococcus, the genus SMB53, the genus Akkermansia, the genus Parabacteroides, the genus Phascolarctobacterium, the genus Catenibacterium, the genus Butyricimonas, the genus Eubacterium, the genus Halomonas, the genus Paraprevotella, the genus Methanobrevibacter, the genus Adlercreutzia, the genus Slackia, the genus Desulfovibrio, and the genus Thermoanaerobacterium was significantly different between atopic dermatitis patients and normal individuals (see Example 5).

According to the result of the exemplary embodiment of the present invention described above, bacteria-derived extracellular vesicles, which are isolated from blood and urine, were compared with those of a normal individual sample through metagenomic analysis, thereby identifying bacteria-derived vesicles, which are significantly changed in content, in an atopic dermatitis patient, and an increase or decrease in content of bacteria-derived vesicles at the above-mentioned level was analyzed through metagenomic analysis, confirming that atopic dermatitis can be diagnosed.

### MODE OF THE INVENTION

Hereinafter, the present invention will be described with reference to exemplary examples to aid in understanding of the present invention. However, these examples are provided only for illustrative purposes and are not intended to limit the scope of the present invention.

### EXAMPLES

### Example 1

### Analysis of In Vivo Absorption, Distribution, and Excretion Patterns of Intestinal Bacteria and Bacteria-Derived Extracellular Vesicles

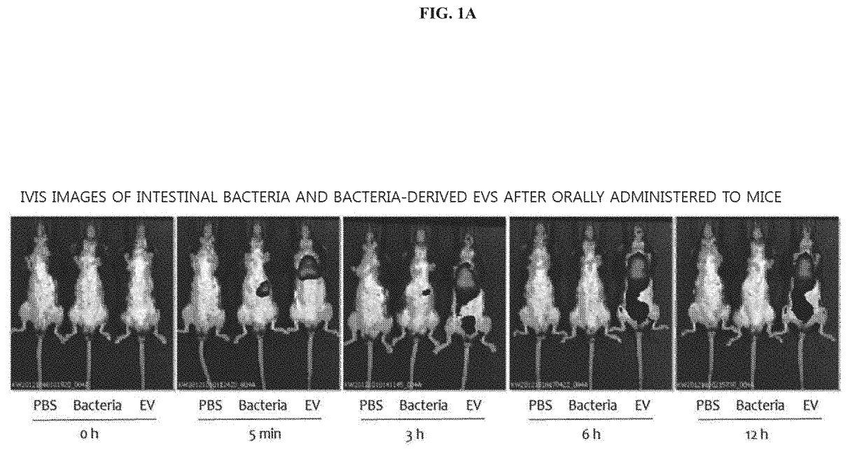

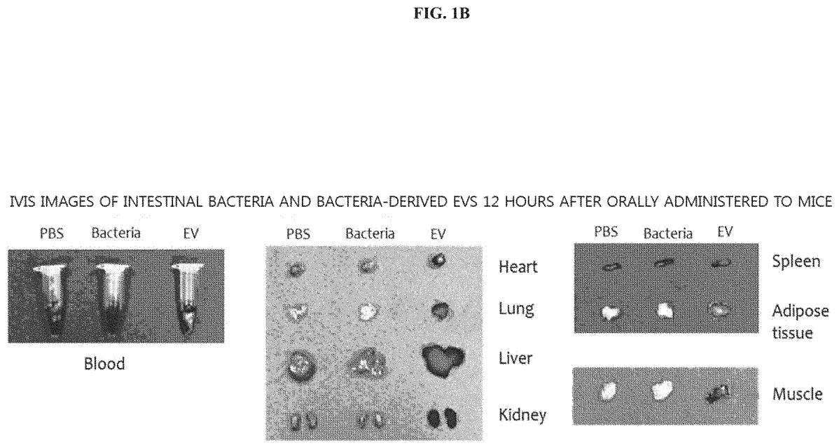

To evaluate whether intestinal bacteria and bacteria-derived extracellular vesicles are systematically absorbed through the gastrointestinal tract, an experiment was conducted using the following method. More particularly, 50 μg of each of intestinal bacteria and the bacteria-derived extracellular vesicles (EVs), labeled with fluorescence, were orally administered to the gastrointestinal tracts of mice, and fluorescence was measured at 0 h, and after 5 min, 3 h, 6 h, and 12 h. As a result of observing the entire images of mice, as illustrated in FIG. 1A, the bacteria were not systematically absorbed when administered, while the bacteria-derived EVs were systematically absorbed at 5 min after administration, and, at 3 h after administration, fluorescence was strongly observed in the bladder, from which it was confirmed that the EVs were excreted via the urinary system, and were present in the bodies up to 12 h after administration.

After intestinal bacteria and intestinal bacteria-derived extracellular vesicles were systematically absorbed, to evaluate a pattern of invasion of intestinal bacteria and the bacteria-derived EVs into various organs in the human body after being systematically absorbed, 50 μg of each of the bacteria and bacteria-derived EVs, labeled with fluorescence, were administered using the same method as that used above, and then, at 12 h after administration, blood, the heart, the lungs, the liver, the kidneys, the spleen, adipose tissue, and muscle were extracted from each mouse. As a result of observing fluorescence in the extracted tissues, as illustrated in FIG. 1B, it was confirmed that the intestinal bacteria were not absorbed into each an, while the bacteria-derived EVs were distributed in the blood, heart, lungs, liver, kidneys, spleen, adipose tissue, and muscle.

### Example 2

### Vesicle Isolation and DNA Extraction from Blood and Urine

To isolate extracellular vesicles and extract DNA, from blood and urine, first, blood or urine was added to a 10 ml tube and centrifuged at 3,500×g and 4° C. for 10 min to precipitate a suspension, and only a supernatant was collected, which was then placed in a new 10 ml tube. The collected supernatant was filtered using a 0.22 μm filter to remove bacteria and impurities, and then placed in centrifugal filters (50 kD) and centrifuged at 1500×g and 4 for 15 min to discard materials with a smaller size than 50 kD, and then concentrated to 10 ml. Once again, bacteria and impurities were removed therefrom using a 0.22 μm filter, and then the resulting concentrate was subjected to ultra-high speed centrifugation at 150,000×g and 4 for 3 hours by using a Type 90ti rotor to remove a supernatant, and the agglomerated pellet was dissolved with phosphate-buffered saline (PBS), thereby obtaining vesicles.

100 μl of the extracellular vesicles isolated from the blood and urine according to the above-described method was boiled at 100 to allow the internal DNA to come out of the lipid and then cooled on ice. Next, the resulting vesicles were centrifuged at 10,000×g and 4 for 30 minutes to remove the remaining suspension, only the supernatant was collected, and then the amount of DNA extracted was quantified using a NanoDrop spectrophotometer. In addition, to verify whether bacteria-derived DNA was present in the extracted DNA, PCR was performed using 16s rDNA primers shown in Table 1 below.

TABLE 1

| Primer | | Sequence | SEQ ID NO. |
|---|---|---|---|
| 16S rDNA | 16_V3_F | 5′-TCGTCGGCAGCGTC | 1 |
| | | AGATGTGTATAAGAG | |
| | | ACAGCCTACGGGNGG | |
| | | CWGCAG-3′ | |
| | 16S_V4_R | 5′-GTCTCGTGGGCTCG | 2 |
| | | GAGATGTGTATAAGA | |
| | | GACAGGACTACHVGG | |
| | | GTATCTAATCC-3′ | |

### Example 3

### Metagenomic Analysis Using DNA Extracted from Blood and Urine

DNA was extracted using the same method as that used in Example 2, and then PCR was performed thereon using 16S rDNA primers shown in Table 1 to amplify DNA, followed by sequencing (Illumina MiSeq sequencer). The results were output as standard flowgram format (SFF) files, and the SFF files were converted into sequence files (.fasta) and nucleotide quality score files using GS FLX software (v2.9), and then credit rating for reads was identified, and portions with a window (20 bps) average base call accuracy of less than 99% (Phred score <20) were removed. After removing the low-quality portions, only reads having a length of 300 bps or more were used (Sickle version 1.33), and, for operational taxonomy unit (OTU) analysis, clustering was performed using UCLUST and USEARCH according to sequence similarity. In particular, clustering was performed based on sequence similarity values of 94% for genus, 90% for family, 85% for order, 80% for class, and 75% for phylum, and phylwn, class, order, family, and genus levels of each OTU were classified, and bacteria with a sequence similarity of 97% or more were analyzed (QIIME) using 16S DNA sequence databases (108,453 sequences) of BLASTN and GreenGenes.

### Example 4

### Atopic Dermatitis Diagnostic Model Based on Metagenomic Analysis of Bacteria-Derived EVs Isolated from Blood

EVs were isolated from blood samples of 25 atopic dermatitis patients and 113 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), accuracy, sensitivity, and specificity, which are diagnostic performance indexes, were calculated by logistic regression analysis.

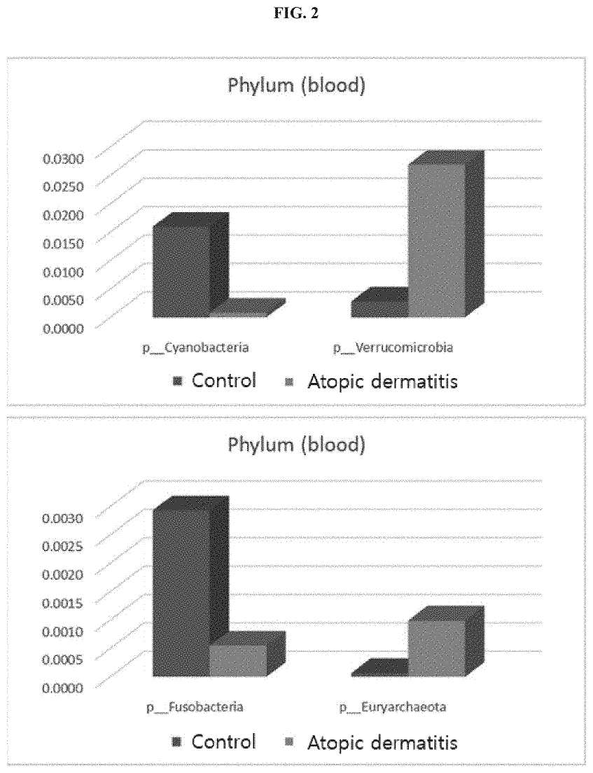

As a result of analyzing bacteria-derived EVs in blood at a phylum level, a diagnostic model developed using bacteria belonging to the phylum Cyanobacteria, the phylum Fusobacteria, the phylum Verrucomicrobia, and the phylum Euryarchaeota as a biomarker exhibited significant diagnostic performance for atopic dermatitis (see Table 2 and FIG. 2).

TABLE 2

| | | | Atopic | | t-test | | | Ac- | Sen- | Spe- |
|---|---|---|---|---|---|---|---|---|---|---|
| | Control | | dermatitis | | p- | | | cur- | sitiv- | cif- |
| Taxon | Mean | SD | Mean | SD | value | Ratio | AUC | acy | ity | city |
| p_Cyano- | 0.0160 | 0.0424 | 0.0008 | 0.0007 | 0.0002 | 0.05 | 0.82 | 0.88 | 1.00 | 0.32 |
| bacteria | | | | | | | | | | |
| p_Fuso- | 0.0029 | 0.0066 | 0.0005 | 0.0005 | 0.0002 | 0.19 | 0.73 | 0.87 | 1.00 | 0.28 |
| bacteria | | | | | | | | | | |
| p_Verruco- | 0.0028 | 0.0059 | 0.0269 | 0.0226 | 0.0000 | 9.54 | 0.88 | 0.90 | 0.96 | 0.60 |
| microbia | | | | | | | | | | |
| p_Euryarch- | 0.0001 | 0.0006 | 0.0010 | 0.0013 | 0.0016 | 14.98 | 0.84 | 0.88 | 0.99 | 0.40 |
| aeota | | | | | | | | | | |

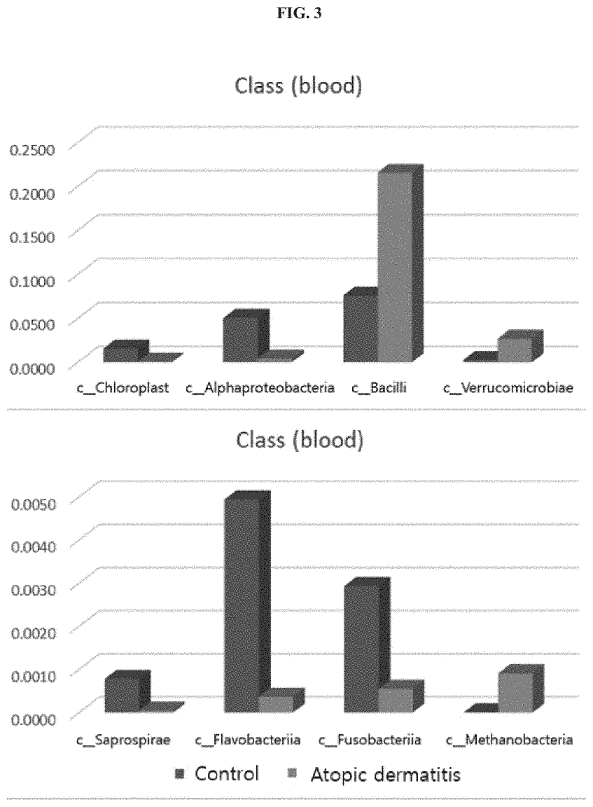

As a result of analyzing bacteria-derived EVs in blood at a class level, a diagnostic model developed using bacteria belonging to the class Chloroplast, the class Saprospirae, the class Flavobacteriia, the class Alphaproteobacteria, the class Fusobacteriia, the class Bacilli, the class Verrucomicrobiae, and the class Methanobacteria as a biomarker exhibited significant diagnostic performance for atopic dermatitis (see Table 3 and FIG. 3).

TABLE 3

| | | | Atopic | | t-test | | | Ac- | Sen- | Spe- |
|---|---|---|---|---|---|---|---|---|---|---|
| | Control | | dermatitis | | p- | | | cur- | sitiv- | cif- |
| Taxon | Mean | SD | Mean | SD | value | Ratio | AUC | acy | ity | city |
| c_Chloro- | 0.0155 | 0.0424 | 0.0007 | 0.0007 | 0.0003 | 0.04 | 0.82 | 0.88 | 1.00 | 0.32 |
| plast | | | | | | | | | | |
| c_Sapro- | 0.0008 | 0.0027 | 0.0000 | 0.0002 | 0.0051 | 0.06 | 0.73 | 0.87 | 1.00 | 0.28 |
| spirae | | | | | | | | | | |
| c_Flavo- | 0.0049 | 0.0105 | 0.0004 | 0.0005 | 0.0000 | 0.07 | 0.77 | 0.87 | 1.00 | 0.28 |
| bacteria | | | | | | | | | | |
| c_Alpha- | 0.0504 | 0.0525 | 0.0042 | 0.0028 | 0.0000 | 0.08 | 0.97 | 0.91 | 0.95 | 0.76 |
| proteo- | | | | | | | | | | |
| bacteria | | | | | | | | | | |
| c_Fuso- | 0.0029 | 0.0066 | 0.0005 | 0.0005 | 0.0002 | 0.19 | 0.73 | 0.87 | 1.00 | 0.28 |
| bacteriia | | | | | | | | | | |
| c_Bacilli | 0.0759 | 0.0468 | 0.2156 | 0.2036 | 0.0022 | 2.84 | 0.78 | 0.89 | 1.00 | 0.40 |
| c_Verru- | 0.0026 | 0.0058 | 0.0268 | 0.0226 | 0.0000 | 10.32 | 0.89 | 0.90 | 0.96 | 0.60 |
| comicrobiae | | | | | | | | | | |
| c_Methano- | 0.0000 | 0.0001 | 0.0009 | 0.0013 | 0.0017 | 110.65 | 0.88 | 0.93 | 0.99 | 0.64 |
| bacteria | | | | | | | | | | |

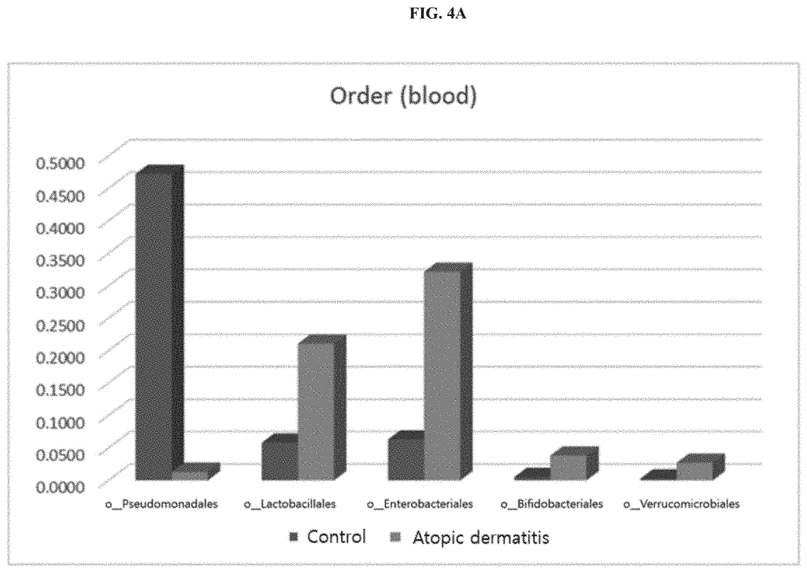

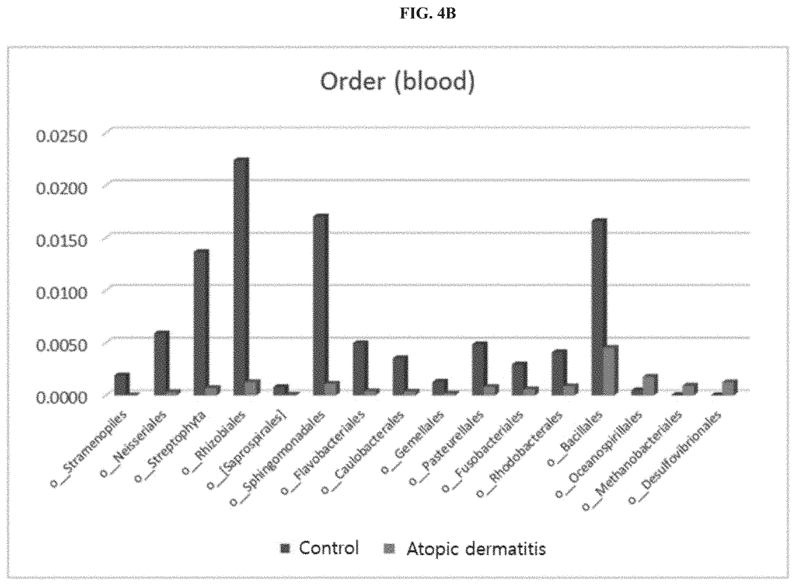

As a result of analyzing bacteria-derived EVs in blood at an order level, a diagnostic model developed using bacteria belonging to the order Stramenopiles, the order Pseudomonadales, the order Neisseriales, the order Streptophyta, the order Rhizobiales, the order Saprospirales, the order Shingomonadales, the order Flavobacteriales, the order Caulobacterales, the order Gemellales, the order Pasteurellales, the order Fusobacteriales, the order Rhodobacterales, the order Bacillales, the order Lactobacillales, the order Oceanospirillales, the order Enterobacteriales, the order Bifidobacteriales, the order Verrucomicrobiales, the order Methanobacteriales, and the order Desulfovibrionales as a biomarker exhibited significant diagnostic performance for atopic dermatitis (see Table 4 and FIGS. 4A and 4B).

TABLE 4

| | | | Atopic | | t-test | | | Ac- | Sen- | Spe- |
|---|---|---|---|---|---|---|---|---|---|---|
| | Control | | dermatitis | | p- | | | cur- | sitiv- | cif- |
| Taxon | Mean | SD | Mean | SD | value | Ratio | AUC | acy | ity | city |
| o_Strameno- | 0.0019 | 0.0061 | 0.000 | 0.0000 | 0.0014 | 0.00 | 0.75 | 0.86 | 1.00 | 0.24 |
| piles | | | | | | | | | | |
| o_Pseudomo- | 0.4721 | 0.1724 | 0.0131 | 0.0087 | 0.0000 | 0.03 | 1.00 | 1.00 | 1.00 | 1.00 |
| nadales | | | | | | | | | | |
| o_Neisseriales | 0.0059 | 0.0142 | 0.0003 | 0.0003 | 0.0001 | 0.05 | 0.78 | 0.87 | 1.00 | 0.28 |
| o_Streptophyta | 0.0136 | 0.0422 | 0.0007 | 0.0007 | 0.0015 | 0.05 | 0.77 | 0.88 | 1.00 | 0.32 |
| o_Rhizobiales | 0.0224 | 0.0273 | 0.0012 | 0.0009 | 0.0000 | 0.06 | 0.98 | 0.94 | 0.96 | 0.88 |
| o_Saprospirales | 0.0008 | 0.0027 | 0.0000 | 0.0002 | 0.0051 | 0.06 | 0.73 | 0.87 | 1.00 | 0.28 |
| o_Sphingo- | 0.0170 | 0.0270 | 0.0011 | 0.0009 | 0.0000 | 0.06 | 0.88 | 0.88 | 0.96 | 0.48 |
| monadales | | | | | | | | | | |
| o_Flavo- | 0.0049 | 0.0105 | 0.0004 | 0.0005 | 0.0000 | 0.07 | 0.77 | 0.87 | 1.00 | 0.28 |
| bacteriales | | | | | | | | | | |
| o_Caulo- | 0.0035 | 0.0103 | 0.0003 | 0.0004 | 0.0014 | 0.09 | 0.75 | 0.86 | 1.00 | 0.24 |
| bacterales | | | | | | | | | | |
| o_Gemellales | 0.0013 | 0.0039 | 0.0002 | 0.0003 | 0.0033 | 0.13 | 0.73 | 0.87 | 1.00 | 0.28 |
| o_Pasteurellales | 0.0048 | 0.0142 | 0.0008 | 0.0009 | 0.0032 | 0.17 | 0.72 | 0.86 | 1.00 | 0.24 |
| o_Fuso- | 0.0029 | 0.0066 | 0.0005 | 0.0005 | 0.0002 | 0.19 | 0.73 | 0.87 | 1.00 | 0.28 |
| bacteriales | | | | | | | | | | |
| o_Rhodo- | 0.0041 | 0.0086 | 0.0008 | 0.0008 | 0.0001 | 0.21 | 0.75 | 0.87 | 1.00 | 0.28 |
| bacterales | | | | | | | | | | |
| o_Bacillales | 0.0166 | 0.0156 | 0.0045 | 0.0030 | 0.0000 | 0.27 | 0.82 | 0.88 | 1.00 | 0.36 |
| o_Lacto- | 0.0578 | 0.0464 | 0.2105 | 0.2021 | 0.0009 | 3.64 | 0.81 | 0.88 | 0.99 | 0.40 |
| bacillales | | | | | | | | | | |
| o_Oceano- | 0.0005 | 0.0018 | 0.0018 | 0.0020 | 0.0021 | 3.78 | 0.74 | 0.86 | 0.98 | 0.32 |
| spirillales | | | | | | | | | | |
| o_Entero- | 0.0629 | 0.0616 | 0.3214 | 0.2882 | 0.0002 | 5.11 | 0.89 | 0.93 | 0.97 | 0.72 |
| bacteriales | | | | | | | | | | |
| o_Bifido- | 0.0049 | 0.0074 | 0.0379 | 0.0421 | 0.0006 | 7.80 | 0.93 | 0.87 | 0.96 | 0.48 |
| bacteriales | | | | | | | | | | |
| o_Verruco- | 0.0026 | 0.0058 | 0.0268 | 0.0226 | 0.0000 | 10.32 | 0.89 | 0.90 | 0.96 | 0.60 |
| microbiales | | | | | | | | | | |
| o_Methano- | 0.0000 | 0.0001 | 0.0009 | 0.0013 | 0.0017 | 110.65 | 0.88 | 0.93 | 0.99 | 0.64 |
| bacteriales | | | | | | | | | | |
| o_Desulf- | 0.0000 | 0.0000 | 0.0012 | 0.0020 | 0.0052 | 744.81 | 0.92 | 0.95 | 0.99 | 0.76 |
| ovibrionales | | | | | | | | | | |

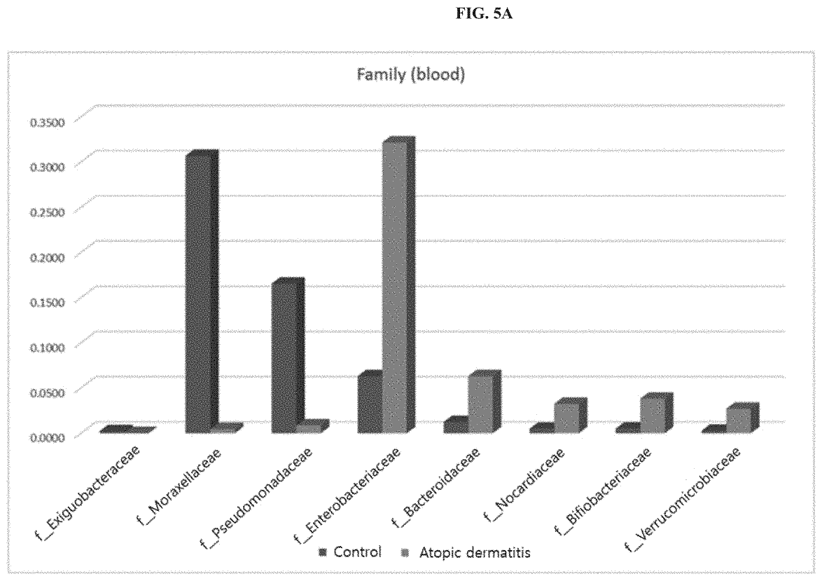

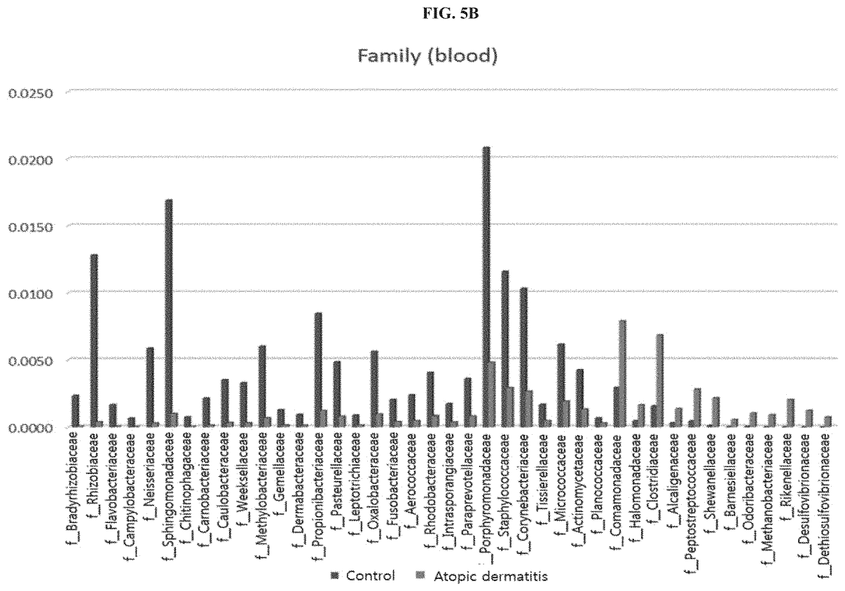

As a result of analyzing bacteria-derived EVs in blood at a family level, a diagnostic model developed using bacteria belonging to the family Exiguobacteraceae, the family Moraxellaceae, the family Bradyrhizobiaceae, the family Rhizobiaceae, the family Flavobacteriaceae, the family Campylobacteraceae, the family Neisseriaceae, the family Pseudomonadaceae, the family Sphingomonadaceae, the family Chitinophagaceae, the family Carnobacteriaceae, the family Caulobacteraceae, the family Weeksellaceae, the family Methylobacteriaceae, the family Gemellaceae, the family Dermabacteraceae, the family Propionibacteriaceae, the family Pasteurellaceae, the family Leptotrichiaceae, the fatly Oxalobacteraceae, the family Fusobacteriaceae, the family Aerococcaceae, the family Rhodobacteraceae, the family Intrasporangiaceae, the family Paraprevotellaceae, the family Porphyromonadaceae, the family Staphylococcaceae, the family Corynebacteriaceae, the family Tissierellaceae, the family Micrococcaceae, the family Actinomycetaceae, the family Planococcaceae, the family Comamonadaceae, the family Halomonadaceae, the family Clostridiaceae, the family Alcaligenaceae, the family Enterobacteriaceae, the family Bacteroidaceae, the family Peptostreptococcaceae, the family Nocardiaceae, the family Bifidobacteriaceae, the family Verrucomicrobiaceae, the family Shewanellaceae, the family Barnesiellaceae, the family Odoribacteraceae, the family Methanobacteriaceae, the family Rikenellaceae, the family Desulfovibrionaceae, and the family Dethiosulfovibrionaceae as a biomarker exhibited significant diagnostic performance for atopic dermatitis (see Table 5 and FIGS. 5A and 5B).

TABLE 5

| | | | Atopic | | t-test | | | Ac- | Sen- | Spe- |
|---|---|---|---|---|---|---|---|---|---|---|
| | Control | | dermatitis | | p- | | | cur- | sitiv- | cif- |
| Taxon | Mean | SD | Mean | SD | value | Ratio | AUC | acy | ity | city |
| f_Exiguobacteraceae | 0.0020 | 0.0077 | 0.0000 | 0.0001 | 0.0068 | 0.01 | 0.73 | 0.86 | 1.00 | 0.24 |
| f_Moraxellaceae | 0.3067 | 0.1632 | 0.0044 | 0.0040 | 0.0000 | 0.01 | 1.00 | 0.99 | 0.99 | 1.00 |
| f_Bradyrhizobiaceae | 0.0023 | 0.0086 | 0.0001 | 0.0001 | 0.0056 | 0.03 | 0.77 | 0.89 | 1.00 | 0.40 |
| f_Rhizobiaceae | 0.0128 | 0.0166 | 0.0004 | 0.0005 | 0.0000 | 0.03 | 0.92 | 0.84 | 0.89 | 0.60 |
| f_Flavobacteriaceae | 0.0017 | 0.0036 | 0.0001 | 0.0001 | 0.0000 | 0.03 | 0.75 | 0.87 | 1.00 | 0.28 |
| f_Campylo- | 0.0007 | 0.0025 | 0.0000 | 0.0001 | 0.0082 | 0.04 | 0.71 | 0.87 | 1.00 | 0.28 |
| bacteraceae | | | | | | | | | | |
| f_Neisseriaceae | 0.0059 | 0.0142 | 0.0003 | 0.0003 | 0.0001 | 0.05 | 0.78 | 0.87 | 1.00 | 0.28 |
| f_Pseudomo- | 0.1652 | 0.1085 | 0.0085 | 0.0063 | 0.0000 | 0.05 | 1.00 | 0.99 | 0.99 | 0.96 |
| nadaceae | | | | | | | | | | |
| f_Sphingo- | 0.0169 | 0.0270 | 0.0010 | 0.0009 | 0.0000 | 0.06 | 0.88 | 0.88 | 0.97 | 0.48 |
| monadaceae | | | | | | | | | | |
| f_Chitino- | 0.0007 | 0.0027 | 0.0000 | 0.0002 | 0.0073 | 0.06 | 0.72 | 0.87 | 1.00 | 0.28 |
| phagaceae | | | | | | | | | | |
| f_Carno- | 0.0021 | 0.0056 | 0.0001 | 0.0002 | 0.0003 | 0.07 | 0.72 | 0.87 | 1.00 | 0.28 |
| bacteriaceae | | | | | | | | | | |
| f_Caulo- | 0.0035 | 0.0103 | 0.0003 | 0.0004 | 0.0014 | 0.09 | 0.75 | 0.86 | 1.00 | 0.24 |
| bacteraceae | | | | | | | | | | |
| f_Weeksellaceae | 0.0033 | 0.0097 | 0.0003 | 0.0004 | 0.0016 | 0.10 | 0.73 | 0.87 | 1.00 | 0.28 |
| f_Methylo- | 0.0060 | 0.0113 | 0.0007 | 0.0008 | 0.0000 | 0.11 | 0.79 | 0.88 | 1.00 | 0.32 |
| bacteriaceae | | | | | | | | | | |
| f_Gemellaceae | 0.0013 | 0.0039 | 0.0002 | 0.0003 | 0.0031 | 0.12 | 0.73 | 0.87 | 1.00 | 0.28 |
| f_Derma- | 0.0009 | 0.0028 | 0.0001 | 0.0003 | 0.0031 | 0.14 | 0.72 | 0.87 | 1.00 | 0.28 |
| bacteraceae | | | | | | | | | | |
| f_Propioni- | 0.0085 | 0.0162 | 0.0012 | 0.0010 | 0.0000 | 0.14 | 0.86 | 0.87 | 0.97 | 0.40 |
| bacteriaceae | | | | | | | | | | |
| f_Pasteurellaceae | 0.0048 | 0.0142 | 0.0008 | 0.0009 | 0.0032 | 0.17 | 0.72 | 0.86 | 1.00 | 0.24 |
| f_Leptotrichiaceae | 0.0009 | 0.0026 | 0.0001 | 0.0002 | 0.0038 | 0.17 | 0.71 | 0.87 | 1.00 | 0.28 |
| f_Oxalo- | 0.0056 | 0.0116 | 0.0010 | 0.0012 | 0.0001 | 0.17 | 0.76 | 0.88 | 1.00 | 0.36 |
| bacteraceae | | | | | | | | | | |
| f_Fuso- | 0.0021 | 0.0053 | 0.0004 | 0.0005 | 0.0014 | 0.20 | 0.71 | 0.87 | 1.00 | 0.28 |
| bacteriaceae | | | | | | | | | | |
| f_Aerococcaceae | 0.0024 | 0.0048 | 0.0005 | 0.0006 | 0.0001 | 0.20 | 0.74 | 0.86 | 1.00 | 0.24 |
| f_Rhodo- | 0.0041 | 0.0086 | 0.0008 | 0.0008 | 0.0002 | 0.21 | 0.75 | 0.87 | 1.00 | 0.28 |
| bacteraceae | | | | | | | | | | |
| f_Intras- | 0.0017 | 0.0042 | 0.0004 | 0.0004 | 0.0010 | 0.21 | 0.73 | 0.86 | 1.00 | 0.24 |
| porangiaceae | | | | | | | | | | |
| f_Paraprevotellaceae | 0.0036 | 0.0099 | 0.0008 | 0.0007 | 0.0038 | 0.22 | 0.71 | 0.86 | 1.00 | 0.24 |
| f_Porphyro- | 0.0208 | 0.0266 | 0.0048 | 0.0038 | 0.0000 | 0.23 | 0.79 | 0.88 | 1.00 | 0.32 |
| monadaceae | | | | | | | | | | |
| f_Staphyl- | 0.0116 | 0.0134 | 0.0029 | 0.0027 | 0.0000 | 0.25 | 0.79 | 0.88 | 1.00 | 0.32 |
| ococcaceae | | | | | | | | | | |
| f_Coryne- | 0.0103 | 0.0111 | 0.0027 | 0.0024 | 0.0000 | 0.26 | 0.80 | 0.87 | 1.00 | 0.28 |
| bacteriaceae | | | | | | | | | | |
| f_Tissierellaceae | 0.0017 | 0.0039 | 0.0005 | 0.0006 | 0.0019 | 0.28 | 0.72 | 0.86 | 1.00 | 0.24 |
| f_Micrococcaceae | 0.0062 | 0.0082 | 0.0019 | 0.0016 | 0.0000 | 0.31 | 0.76 | 0.87 | 1.00 | 0.28 |
| f_Actino- | 0.0043 | 0.0097 | 0.0013 | 0.0015 | 0.0028 | 0.31 | 0.71 | 0.86 | 1.00 | 0.24 |
| mycetaceae | | | | | | | | | | |
| f_Planococcaceae | 0.0007 | 0.0013 | 0.0003 | 0.0003 | 0.0073 | 0.44 | 0.71 | 0.87 | 1.00 | 0.28 |
| f_Comamo- | 0.0029 | 0.0070 | 0.0079 | 0.0091 | 0.0030 | 2.68 | 0.75 | 0.87 | 0.99 | 0.32 |
| nadaceae | | | | | | | | | | |
| f_Halomo- | 0.0005 | 0.0018 | 0.0016 | 0.0020 | 0.0044 | 3.57 | 0.72 | 0.85 | 0.98 | 0.24 |
| nadaceae | | | | | | | | | | |
| f_Clostridiaceae | 0.0016 | 0.0035 | 0.0069 | 0.0092 | 0.0094 | 4.36 | 0.76 | 0.87 | 0.97 | 0.40 |
| f_Alcaligenaceae | 0.0003 | 0.0015 | 0.0014 | 0.0015 | 0.0020 | 4.36 | 0.77 | 0.87 | 0.99 | 0.32 |
| f_Entero- | 0.0629 | 0.0616 | 0.3214 | 0.2882 | 0.0002 | 5.11 | 0.89 | 0.93 | 0.97 | 0.72 |
| bacteriaceae | | | | | | | | | | |
| f_Bacteroidaceae | 0.0122 | 0.0226 | 0.0629 | 0.0460 | 0.0000 | 5.15 | 0.87 | 0.91 | 0.98 | 0.56 |
| f_Pepto- | 0.0004 | 0.0017 | 0.0028 | 0.0028 | 0.0003 | 6.33 | 0.79 | 0.88 | 0.98 | 0.44 |
| streptococcaceae | | | | | | | | | | |
| f_Nocardiaceae | 0.0048 | 0.0112 | 0.0323 | 0.0408 | 0.0026 | 6.78 | 0.79 | 0.88 | 0.98 | 0.40 |
| f_Bifido- | 0.0049 | 0.0074 | 0.0379 | 0.0421 | 0.0006 | 7.80 | 0.93 | 0.87 | 0.96 | 0.48 |
| bacteriaceae | | | | | | | | | | |
| f_Verrucomicro- | 0.0026 | 0.0058 | 0.0268 | 0.0226 | 0.0000 | 10.32 | 0.89 | 0.90 | 0.96 | 0.60 |
| biaceae | | | | | | | | | | |
| f_Shewanellaceae | 0.0001 | 0.0007 | 0.0022 | 0.0041 | 0.0207 | 18.17 | 0.78 | 0.88 | 0.97 | 0.48 |
| f_Barnesiellaceae | 0.0000 | 0.0003 | 0.0006 | 0.0006 | 0.0004 | 21.84 | 0.87 | 0.90 | 0.99 | 0.48 |
| f_Odoribacteraceae | 0.0000 | 0.0003 | 0.0010 | 0.0014 | 0.0021 | 22.59 | 0.84 | 0.88 | 0.97 | 0.48 |
| f_Methano- | 0.0000 | 0.0001 | 0.0009 | 0.0013 | 0.0017 | 110.65 | 0.88 | 0.93 | 0.99 | 0.64 |
| bacteriaceae | | | | | | | | | | |
| f_Rikenellaceae | 0.0000 | 0.0001 | 0.0020 | 0.0020 | 0.0000 | 122.32 | 0.94 | 0.93 | 0.98 | 0.72 |
| f_Desulfo- | 0.0000 | 0.0000 | 0.0012 | 0.0020 | 0.0052 | 744.81 | 0.92 | 0.95 | 0.99 | 0.76 |
| vibrionaceae | | | | | | | | | | |
| f_Dethiosulfo- | 0.0000 | 0.0000 | 0.0007 | 0.0017 | 0.0000 | | 0.93 | 0.96 | 1.00 | 0.80 |
| vibrionaceae | | | | | | | | | | |

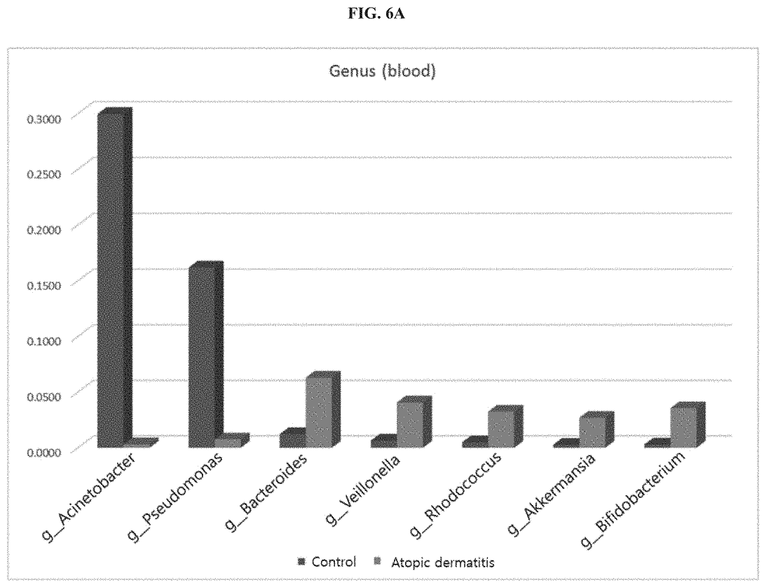

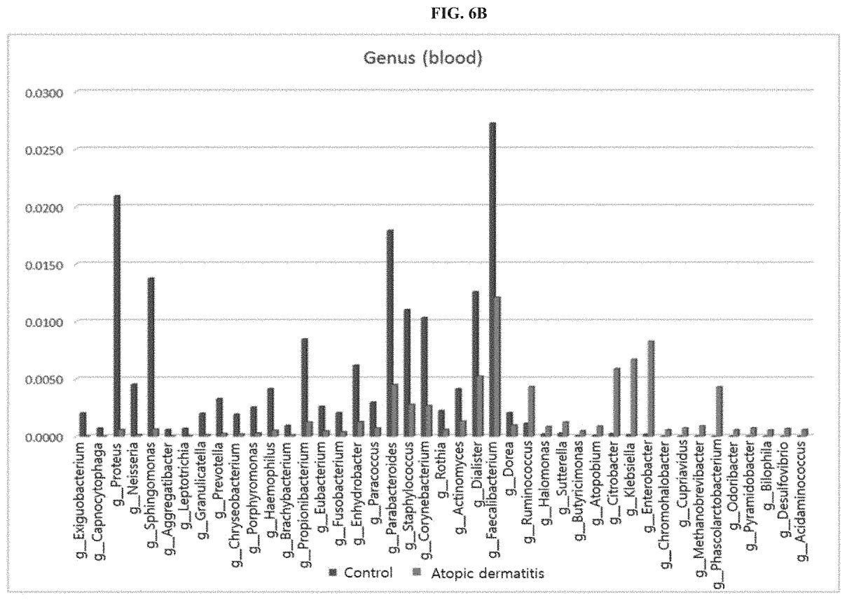

As a result of analyzing bacteria-derived EVs in blood at a genus level, a diagnostic model developed using bacteria belonging to the genus Exiguobacterium, the genus Acinetobacter, the genus Capnocytophaga, the genus Proteus, the genus Neisseria, the genus Sphingomonas, the genus Pseudomonas, the genus Aggregatibacter, the genus Leptotrichia, the genus Granulicatella, the genus Prevotella, the genus Chryseobacterium, the genus Porphyromonas, the genus Haemophilus, the genus Brachybacterium, the genus Propionibacterium, the genus Eubacterium, the genus Fusobacterium, the genus Enhydrobacter, the genus Paracoccus, the genus Parabacteroides, the genus Staphylococcus, the genus Corynebacterium, the genus Rothia, the genus Actinomyces, the genus Dialister, the genus Faecalibacterium, the genus Dorea, the genus Ruminococcus, the genus Halomonas, the genus Sutterella, the genus Bacteroides, the genus Veillonella, the genus Rhodococcus, the genus Butyricimonas, the genus Akkermansia, the genus Bifidobacterium, the genus Atopobium, the genus Citrobacter, the genus Klebsiella, the genus Enterobacter, the genus Chromohalobacter, the genus Cupriavidus, the genus Methanobrevibacter, the genus Phascolarctobacterium, the genus Odoribacter, the genus Pyramidobacter, the genus Bilophila, the genus Desulfovibrio, and the genus Acidaminococcus as a biomarker exhibited significant diagnostic performance for atopic dermatitis (see Table 6 and FIGS. 6A and 6B).

TABLE 6

| | | | Atopic | | t-test | | | Ac- | Sen- | Spe- |
|---|---|---|---|---|---|---|---|---|---|---|
| | Control | | dermatitis | | p- | | | cur- | sitiv- | cif- |
| Taxon | Mean | SD | Mean | SD | value | Ratio | AUC | acy | ity | city |
| g_Exiguobacterium | 0.0020 | 0.0077 | 0.0000 | 0.0000 | 0.0067 | 0.01 | 0.73 | 0.86 | 1.00 | 0.24 |
| g_Acinetobacter | 0.2996 | 0.1634 | 0.0030 | 0.0030 | 0.0000 | 0.01 | 1.00 | 0.99 | 0.99 | 1.00 |
| g_Capnocytophaga | 0.0007 | 0.0027 | 0.0000 | 0.0000 | 0.0066 | 0.01 | 0.72 | 0.87 | 1.00 | 0.28 |
| g_Proteus | 0.0209 | 0.0264 | 0.0006 | 0.0006 | 0.0000 | 0.03 | 0.91 | 0.88 | 0.96 | 0.48 |
| g_Neisseria | 0.0045 | 0.0132 | 0.0001 | 0.0002 | 0.0006 | 0.03 | 0.76 | 0.87 | 1.00 | 0.28 |
| g_Sphingomonas | 0.0138 | 0.0265 | 0.0006 | 0.0006 | 0.0000 | 0.05 | 0.86 | 0.87 | 0.96 | 0.44 |
| g_Pseudomonas | 0.1616 | 0.1075 | 0.0075 | 0.0055 | 0.0000 | 0.05 | 1.00 | 0.99 | 0.99 | 0.96 |
| g_Aggregatibacter | 0.0006 | 0.0018 | 0.0000 | 0.0001 | 0.0010 | 0.05 | 0.73 | 0.86 | 1.00 | 0.24 |
| g_Leptotrichia | 0.0007 | 0.0022 | 0.0000 | 0.0001 | 0.0021 | 0.06 | 0.72 | 0.87 | 1.00 | 0.28 |
| g_Granulicatella | 0.0020 | 0.056 | 0.0001 | 0.0002 | 0.0006 | 0.07 | 0.72 | 0.87 | 1.00 | 0.28 |
| g_Prevotella | 0.0033 | 0.0098 | 0.0002 | 0.0003 | 0.0013 | 0.07 | 0.72 | 0.87 | 1.00 | 0.28 |
| g_Chryseobacterium | 0.0019 | 0.0067 | 0.0002 | 0.0003 | 0.0074 | 0.10 | 0.72 | 0.87 | 1.00 | 0.28 |
| g_Porphyromonas | 0.0025 | 0.0070 | 0.0003 | 0.0006 | 0.0011 | 0.11 | 0.75 | 0.87 | 1.00 | 0.28 |
| g_Haemophilus | 0.0041 | 0.0132 | 0.0005 | 0.0006 | 0.0045 | 0.13 | 0.72 | 0.86 | 1.00 | 0.24 |
| g_Brachybacterium | 0.0009 | 0.0028 | 0.0001 | 0.0003 | 0.0031 | 0.14 | 0.72 | 0.87 | 1.00 | 0.28 |
| g_Propionibacterium | 0.0085 | 0.0162 | 0.0012 | 0.0010 | 0.0000 | 0.14 | 0.86 | 0.87 | 0.97 | 0.40 |
| g_Eubacterium | 0.0026 | 0.0038 | 0.0005 | 0.0006 | 0.0000 | 0.18 | 0.79 | 0.88 | 1.00 | 0.32 |
| g_Fusobacterium | 0.0021 | 0.0053 | 0.0004 | 0.0005 | 0.0013 | 0.19 | 0.71 | 0.87 | 1.00 | 0.28 |
| g_Enhydrobacter | 0.0062 | 0.0189 | 0.0013 | 0.0012 | 0.0071 | 0.20 | 0.74 | 0.86 | 1.00 | 0.24 |
| g_Paracoccus | 0.0030 | 0.0070 | 0.0007 | 0.0008 | 0.0012 | 0.24 | 0.73 | 0.87 | 1.00 | 0.28 |
| g_Parabacteroides | 0.0179 | 0.0260 | 0.0045 | 0.0038 | 0.0000 | 0.25 | 0.76 | 0.86 | 1.00 | 0.24 |
| g_Staphylococcus | 0.0110 | 0.0134 | 0.0028 | 0.0025 | 0.0000 | 0.25 | 0.77 | 0.88 | 1.00 | 0.32 |
| g_Corynebacterium | 0.0103 | 0.0111 | 0.0027 | 0.0024 | 0.0000 | 0.26 | 0.80 | 0.87 | 1.00 | 0.28 |
| g_Rothia | 0.0022 | 0.0053 | 0.0006 | 0.0008 | 0.0022 | 0.27 | 0.72 | 0.86 | 1.00 | 0.24 |
| g_Actinomyces | 0.0041 | 0.0094 | 0.0013 | 0.0015 | 0.0031 | 0.32 | 0.71 | 0.86 | 1.00 | 0.24 |
| g_Dialister | 0.0126 | 0.0141 | 0.0052 | 0.0052 | 0.0000 | 0.42 | 0.76 | 0.87 | 1.00 | 0.28 |
| g_Faecalibacterium | 0.0273 | 0.0270 | 0.0121 | 0.0089 | 0.0000 | 0.44 | 0.74 | 0.87 | 1.00 | 0.28 |
| g_Dorea | 0.0021 | 0.0038 | 0.0010 | 0.0007 | 0.0065 | 0.48 | 0.71 | 0.86 | 1.00 | 0.24 |
| g_Ruminococcus | 0.0011 | 0.0025 | 0.0043 | 0.0039 | 0.0005 | 3.79 | 0.78 | 0.88 | 0.99 | 0.40 |
| g_Halomonas | 0.0002 | 0.0010 | 0.0008 | 0.0011 | 0.0032 | 4.42 | 0.71 | 0.87 | 0.99 | 0.32 |
| g_Sutterella | 0.0003 | 0.0015 | 0.0012 | 0.0016 | 0.0038 | 4.68 | 0.77 | 0.86 | 0.99 | 0.28 |
| g_Bacteroides | 0.0122 | 0.0226 | 0.0629 | 0.0460 | 0.0000 | 5.15 | 0.87 | 0.91 | 0.98 | 0.56 |
| g_Veillonella | 0.0064 | 0.0123 | 0.0406 | 0.0576 | 0.0068 | 6.33 | 0.79 | 0.88 | 0.98 | 0.40 |
| g_Rhodococcus | 0.0048 | 0.0112 | 0.0323 | 0.0408 | 0.0026 | 6.78 | 0.79 | 0.88 | 0.98 | 0.40 |
| g_Butyricimonas | 0.0000 | 0.0003 | 0.0005 | 0.0006 | 0.0024 | 10.26 | 0.78 | 0.86 | 0.97 | 0.36 |
| g_Akkermansia | 0.0026 | 0.0058 | 0.0267 | 0.0225 | 0.0000 | 10.28 | 0.89 | 0.90 | 0.96 | 0.60 |
| g_Bifidobacterium | 0.0028 | 0.0052 | 0.0354 | 0.0434 | 0.0010 | 1.244 | 0.89 | 0.89 | 0.96 | 0.56 |
| g_Atopobium | 0.0001 | 0.0005 | 0.0009 | 0.0012 | 0.0022 | 14.44 | 0.85 | 0.88 | 0.99 | 0.40 |
| g_Citrobacter | 0.0002 | 0.0010 | 0.0059 | 0.0055 | 0.0000 | 25.46 | 0.84 | 0.91 | 0.99 | 0.56 |
| g_Klebsiella | 0.0002 | 0.0009 | 0.0067 | 0.0069 | 0.0001 | 44.31 | 0.86 | 0.93 | 0.99 | 0.68 |
| g_Enterobacter | 0.0002 | 0.0013 | 0.0083 | 0.0109 | 0.0011 | 46.60 | 0.87 | 0.91 | 0.98 | 0.60 |
| g_Chromohalobacter | 0.0000 | 0.0001 | 0.0006 | 0.0009 | 0.0038 | 52.80 | 0.85 | 0.90 | 0.99 | 0.48 |
| g_Cupriavidus | 0.0000 | 0.0000 | 0.0007 | 0.0012 | 0.0060 | 73.97 | 0.87 | 0.94 | 0.99 | 0.72 |
| g_Methanobrevibacter | 0.0000 | 0.0001 | 0.0009 | 0.0013 | 0.0017 | 109.16 | 0.88 | 0.93 | 0.99 | 0.64 |
| g_Phasco- | 0.0000 | 0.0000 | 0.0043 | 0.0047 | 0.0001 | 6370.88 | 0.97 | 0.99 | 1.00 | 0.92 |
| larctobacterium | | | | | | | | | | |
| g_Odoribacter | 0.0000 | 0.0000 | 0.0006 | 0.0010 | 0.0000 | | 0.95 | 0.97 | 1.00 | 0.84 |
| g_Pyramidobacter | 0.0000 | 0.0000 | 0.0007 | 0.0017 | 0.0000 | | 0.94 | 0.96 | 1.00 | 0.76 |
| g_Bilophila | 0.0000 | 0.0000 | 0.0005 | 0.0007 | 0.0000 | | 0.91 | 0.96 | 1.00 | 0.76 |
| g_Desulfovibrio | 0.0000 | 0.0000 | 0.0007 | 0.0020 | 0.0003 | | 0.87 | 0.93 | 1.00 | 0.64 |
| g_Acidamino- | 0.0000 | 0.0000 | 0.0006 | 0.0015 | 0.0001 | | 0.85 | 0.91 | 1.00 | 0.52 |
| coccus | | | | | | | | | | |

### Example 5

### Atopic Dermatitis Diagnostic Model Based on Metagenomic Analysis of Bacteria-Derived EVs Isolated from Urine

EVs were isolated from urine samples of 59 atopic dermatitis patients and 98 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), sensitivity, and specificity, which are diagnostic performance indexes, were calculated by logistic regression analysis.

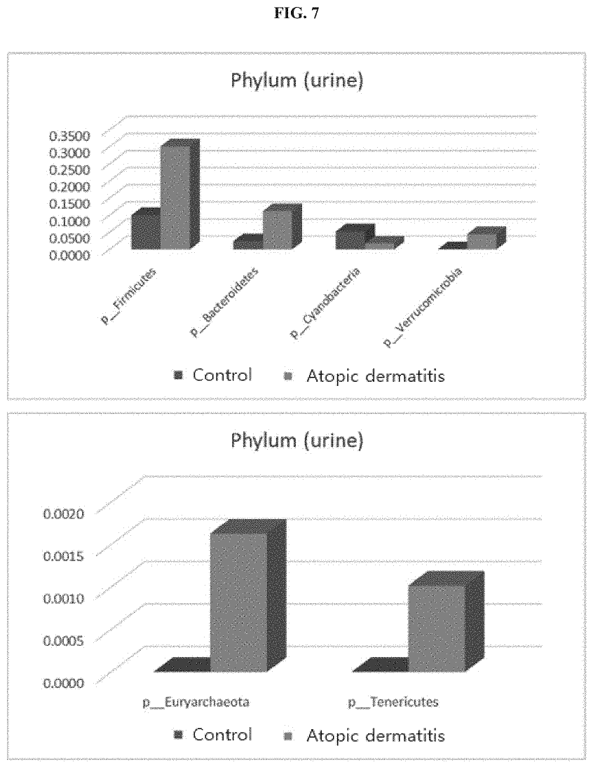

As a result of analyzing bacteria-derived EVs in urine at a phylum level, a diagnostic model developed using bacteria belonging to the phylum Cyanobacteria, the phylum Firmicutes, the phylum Bacteroidetes, the phylum Verrucomicrobia, the phylum Euryarchaeota, and the phylum Tenericutes as a biomarker exhibited significant diagnostic performance for atopic dermatitis (see Table 7 and FIG. 7).

TABLE 7

| | | | Atopic | | | | | Ac- | Sen- | Spe- |
|---|---|---|---|---|---|---|---|---|---|---|
| | Control | | dermatitis | | t-test | | | cur- | sitiv- | cif- |
| Taxon | Mean | SD | Mean | SD | p-value | Ratio | AUC | acy | ity | icity |
| p_Cyanobacteria | 0.0522 | 0.0460 | 0.0166 | 0.0316 | 0.0000 | 0.32 | 0.75 | 0.73 | 0.44 | 0.91 |
| p_Firmicutes | 0.1003 | 0.0870 | 0.3015 | 0.1767 | 0.0000 | 3.00 | 0.81 | 0.72 | 0.73 | 0.71 |
| p_Bacteroidetes | 0.0240 | 0.0408 | 0.1115 | 0.0847 | 0.0000 | 4.65 | 0.80 | 0.75 | 0.81 | 0.70 |
| p_Verrucomicrobia | 0.0002 | 0.0016 | 0.0447 | 0.0500 | 0.0000 | 209.30 | 0.84 | 0.77 | 0.98 | 0.64 |
| p_Euryarchaeota | 0.0000 | 0.0000 | 0.0016 | 0.0027 | 0.0000 | | 0.77 | 0.68 | 1.00 | 0.49 |
| p_Tenericutes | 0.0000 | 0.0000 | 0.0010 | 0.0016 | 0.0000 | | 0.76 | 0.71 | 1.00 | 0.53 |

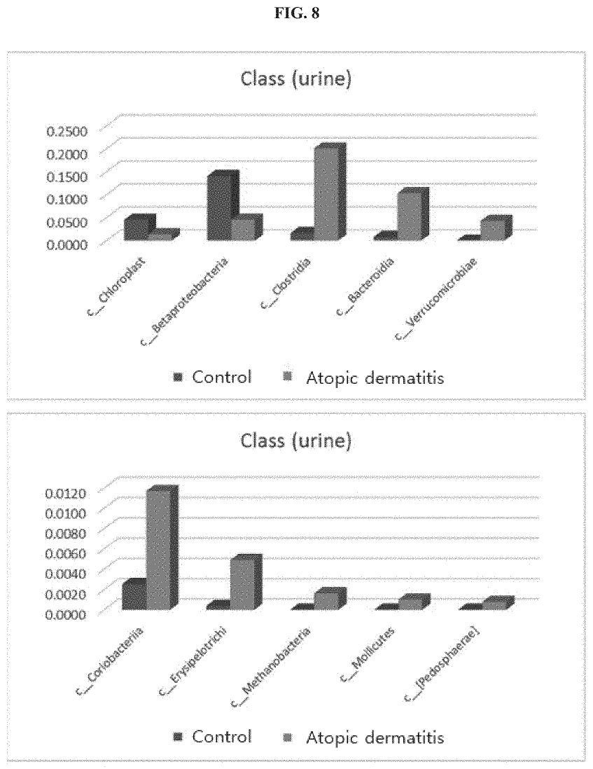

As a result of analyzing bacteria-derived EVs in urine at a class level, a diagnostic model developed using bacteria belonging to the class Chloroplast, the class Betaproteobacteria, the class Coriobacteriia, the class Clostridia, the class Bacteroidia, the class Erysipelotrichi, the class Verrucomicrobiae, the class Methanobacteria, the class Mollicutes, and the class Pedosphaerae as a biomarker exhibited significant diagnostic performance for atopic dermatitis (see Table 8 and FIG. 8).

TABLE 8

| | | | Atopic | | | | | Ac- | Sen- | Spe- |
|---|---|---|---|---|---|---|---|---|---|---|
| | Control | | dermatitis | | t-test | | | cur- | sitiv- | cif- |
| Taxon | Mean | SD | Mean | SD | p-value | Ratio | AUC | acy | ity | icity |
| c_Chloroplast | 0.0457 | 0.0427 | 0.0137 | 0.0252 | 0.0000 | 0.30 | 0.75 | 0.73 | 0.44 | 0.90 |
| c_Betaproteobacteria | 0.1406 | 0.0881 | 0.0457 | 0.0439 | 0.0000 | 0.32 | 0.86 | 0.78 | 0.61 | 0.88 |
| c_Coriobacteriia | 0.0025 | 0.0127 | 0.0117 | 0.0113 | 0.0000 | 4.62 | 0.79 | 0.74 | 0.88 | 0.65 |
| c_Clostridia | 0.0165 | 0.0398 | 0.2010 | 0.1599 | 0.0000 | 12.19 | 0.85 | 0.73 | 0.90 | 0.63 |
| c_Bacteroidia | 0.0082 | 0.0295 | 0.1031 | 0.0886 | 0.0000 | 12.62 | 0.85 | 0.76 | 0.90 | 0.67 |
| c_Erysipelotrichi | 0.0004 | 0.0027 | 0.0049 | 0.0050 | 0.0000 | 13.63 | 0.81 | 0.75 | 0.98 | 0.61 |
| c_Verrucomicrobiae | 0.0000 | 0.0000 | 0.0430 | 0.0482 | 0.0000 | | 0.83 | 0.78 | 1.00 | 0.64 |
| c_Methanobacteria | 0.0000 | 0.0000 | 0.0016 | 0.0027 | 0.0000 | | 0.77 | 0.68 | 1.00 | 0.49 |
| c_Mollicutes | 0.0000 | 0.0000 | 0.0010 | 0.0015 | 0.0000 | | 0.76 | 0.71 | 1.00 | 0.53 |
| c_Pedosphaerae | 0.0000 | 0.0000 | 0.0008 | 0.0014 | 0.0001 | | 0.70 | 0.62 | 0.31 | 0.82 |

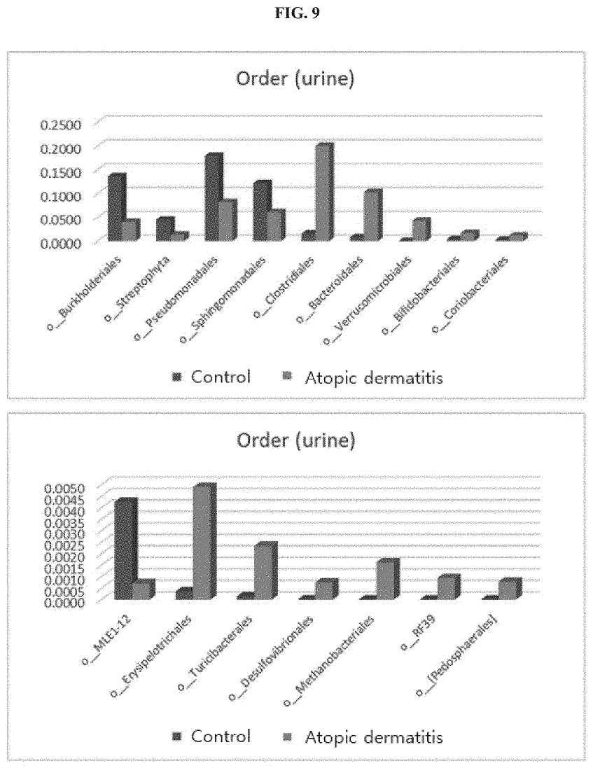

As a result of analyzing bacteria-derived EVs in urine at an order level, a diagnostic model developed using bacteria belonging to the order MLE1-12, the order Burkholderiales, the order Streptophyta, the order Pseudomonadales, the order Sphingomonadales, the order Bifidobacteriales, the order Coriobacteriales, the order Clostridiales, the order Bacteroidales, the order Erysipelotrichales, the order Turicibacterales, the order Desulfovibrionales, the order Verrucomicrobiales, the order Methanobacteriales, the order RF39, and the order Pedosphaerales as a biomarker exhibited significant diagnostic performance for atopic dermatitis (see Table 9 and FIG. 9).

TABLE 9

| | | | Atopic | | | | | Ac- | Sen- | Spe- |
|---|---|---|---|---|---|---|---|---|---|---|
| | Control | | dermatitis | | t-test | | | cur- | sitiv- | cif- |
| Taxon | Mean | SD | Mean | SD | p-value | Ratio | AUC | acy | ity | icity |
| o_MLE1-12 | 0.0043 | 0.0096 | 0.0007 | 0.0036 | 0.0088 | 0.17 | 0.66 | 0.69 | 0.20 | 0.98 |
| o_Burkholderiales | 0.1360 | 0.0874 | 0.0406 | 0.0419 | 0.0000 | 0.30 | 0.87 | 0.76 | 0.63 | 0.85 |
| o_Streptophyta | 0.0454 | 0.0425 | 0.0137 | 0.0252 | 0.0000 | 0.30 | 0.75 | 0.73 | 0.44 | 0.90 |
| o_Pseudomonadales | 0.1787 | 0.1129 | 0.0819 | 0.0746 | 0.0000 | 0.46 | 0.77 | 0.68 | 0.46 | 0.82 |
| o_Sphingomonadales | 0.1219 | 0.0569 | 0.0607 | 0.0811 | 0.0000 | 0.50 | 0.74 | 0.63 | 0.42 | 0.76 |
| o_Bifidobacteriales | 0.0043 | 0.0091 | 0.0172 | 0.0136 | 0.0000 | 4.02 | 0.75 | 0.71 | 0.76 | 0.68 |
| o_Coriobacteriales | 0.0025 | 0.0127 | 0.0117 | 0.0113 | 0.0000 | 4.62 | 0.79 | 0.74 | 0.88 | 0.65 |
| o_Clostridiales | 0.0163 | 0.0397 | 0.2006 | 0.1599 | 0.0000 | 12.28 | 0.85 | 0.73 | 0.88 | 0.63 |
| o_Bacteroidales | 0.0082 | 0.0295 | 0.1031 | 0.0886 | 0.0000 | 12.62 | 0.85 | 0.76 | 0.90 | 0.67 |
| o_Erysipelotrichales | 0.0004 | 0.0027 | 0.0049 | 0.0050 | 0.0000 | 13.63 | 0.81 | 0.75 | 0.98 | 0.61 |
| o_Turicibacterales | 0.0001 | 0.0009 | 0.0023 | 0.0029 | 0.0000 | 16.28 | 0.79 | 0.72 | 0.97 | 0.57 |
| o_Desulfovibrionales | 0.0000 | 0.0000 | 0.0007 | 0.0015 | 0.0000 | 503.89 | 0.78 | 0.68 | 0.98 | 0.49 |
| o_Verrucomicrobiales | 0.0000 | 0.0000 | 0.0430 | 0.0482 | 0.0000 | | 0.83 | 0.78 | 1.00 | 0.64 |
| o_Methanobacteriales | 0.0000 | 0.0000 | 0.0016 | 0.0027 | 0.0000 | | 0.77 | 0.69 | 1.00 | 0.49 |
| o_RF39 | 0.0000 | 0.0000 | 0.0009 | 0.0014 | 0.0000 | | 0.76 | 0.71 | 1.00 | 0.53 |
| o_Pedosphaerales | 0.0000 | 0.0000 | 0.0008 | 0.0014 | 0.0001 | | 0.70 | 0.62 | 0.31 | 0.82 |

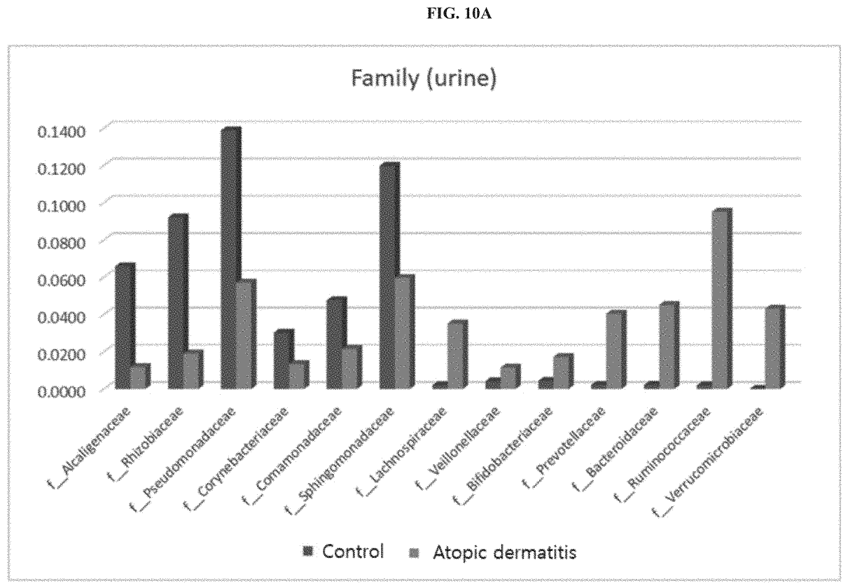

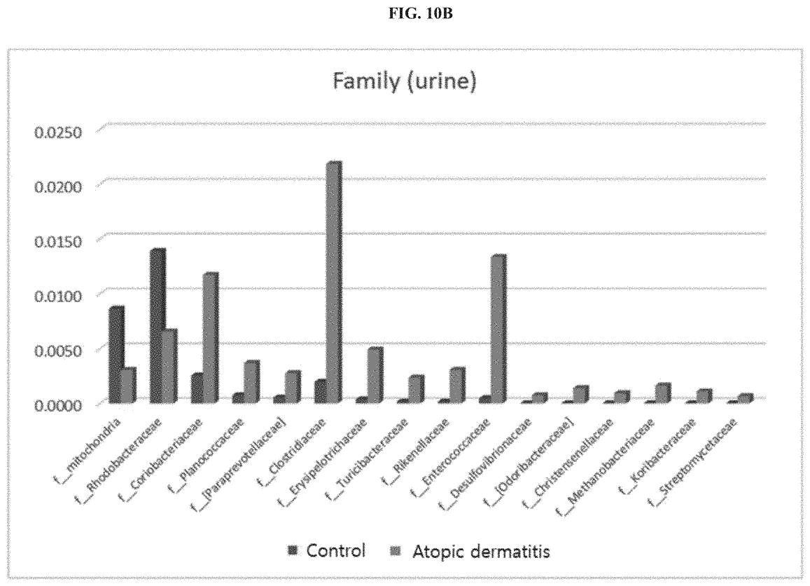

As a result of analyzing bacteria-derived EVs in urine at a family level, a diagnostic model developed using bacteria belonging to the family Alcaligenaceae, the family Rhizobiaceae, the family mitochondria, the family Pseudomonadaceae, the family Corynebacteriaceae, the family Comamonadaceae, the family Rhodobacteraceae, the family Sphingomonadaceae, the family Veillonellaceae, the family Bifidobacteriaceae, the family Coriobacteriaceae, the family Planococcaceae, the family Paraprevotellaceae, the family Clostridiaceae, the family Erysipelotrichaceae, the family Turicibacteraceae, the family Lachnospiraceae, the family Prevotellaceae, the family Rikenellaceae, the family Bacteroidaceae, the family Enterococcaceae, the family Ruminococcaceae, the family Desulfovibrionaceae, the family Verrucomicrobiaceae, the family Odoribacteraceae, the family Christensenellaceae, the family Methanobacteriaceae, the family Koribacteraceae, and the family Streptomycetaceae as a biomarker exhibited significant diagnostic performance for atopic dermatitis (see Table 10 and FIGS. 10A and 10B).

TABLE 10

| | | | Atopic | | | | | Ac- | Sen- | Spe- |
|---|---|---|---|---|---|---|---|---|---|---|
| | Control | | dermatitis | | t-test | | | cur- | sitiv- | cif- |
| Taxon | Mean | SD | Mean | SD | p-value | Ratio | AUC | acy | ity | icity |
| f_Alcaligenaceae | 0.0658 | 0.0694 | 0.0117 | 0.0216 | 0.0000 | 0.18 | 0.80 | 0.76 | 0.58 | 0.87 |
| f_Rhizobiaceae | 0.0921 | 0.0717 | 0.0190 | 0.0353 | 0.0000 | 0.21 | 0.87 | 0.80 | 0.64 | 0.89 |
| f_mitochondria | 0.0086 | 0.0145 | 0.0030 | 0.0080 | 0.0085 | 0.35 | 0.65 | 0.63 | 0.19 | 0.90 |
| f_Pseudomonadaceae | 0.1388 | 0.0923 | 0.0570 | 0.0574 | 0.0000 | 0.41 | 0.80 | 0.67 | 0.42 | 0.82 |
| f_Corynebacteriaceae | 0.0302 | 0.0234 | 0.0134 | 0.0164 | 0.0000 | 0.44 | 0.75 | 0.71 | 0.42 | 0.88 |
| f_Comamonadaceae | 0.0476 | 0.0405 | 0.0215 | 0.0275 | 0.0000 | 0.45 | 0.75 | 0.66 | 0.31 | 0.87 |
| f_Rhodobacteraceae | 0.0139 | 0.0188 | 0.0065 | 0.0123 | 0.0095 | 0.47 | 0.63 | 0.64 | 0.20 | 0.90 |
| f_Sphingomonadaceae | 0.1198 | 0.0571 | 0.0596 | 0.0799 | 0.0000 | 0.50 | 0.74 | 0.63 | 0.42 | 0.76 |
| f_Veillonellace | 0.0041 | 0.0107 | 0.0114 | 0.0105 | 0.0000 | 2.77 | 0.74 | 0.69 | 0.56 | 0.78 |
| f_Bifidobacteriaceae | 0.0043 | 0.0091 | 0.0172 | 0.0136 | 0.0000 | 4.02 | 0.75 | 0.71 | 0.76 | 0.68 |
| f_Coriobacteriaceae | 0.0025 | 0.0127 | 0.0117 | 0.0113 | 0.0000 | 4.62 | 0.79 | 0.74 | 0.88 | 0.65 |
| f_Planococcaceae | 0.0007 | 0.0021 | 0.0037 | 0.0074 | 0.0004 | 4.99 | 0.74 | 0.62 | 0.02 | 0.99 |
| f_Paraprevotellaceae | 0.0005 | 0.0039 | 0.0027 | 0.0044 | 0.0019 | 5.31 | 0.79 | 0.64 | 0.15 | 0.93 |
| f_Clostridiaceae | 0.0020 | 0.0103 | 0.0218 | 0.0207 | 0.0000 | 11.17 | 0.81 | 0.76 | 0.93 | 0.65 |
| f_Erysipelotrichaceae | 0.0004 | 0.0027 | 0.0049 | 0.0050 | 0.0000 | 13.63 | 0.81 | 0.75 | 0.98 | 0.61 |
| f_Turicibacteraceae | 0.0001 | 0.0009 | 0.0023 | 0.0029 | 0.0000 | 16.28 | 0.79 | 0.72 | 0.97 | 0.57 |
| f_Lachnospiraceae | 0.0020 | 0.0120 | 0.0351 | 0.0367 | 0.0000 | 17.25 | 0.83 | 0.76 | 0.95 | 0.64 |

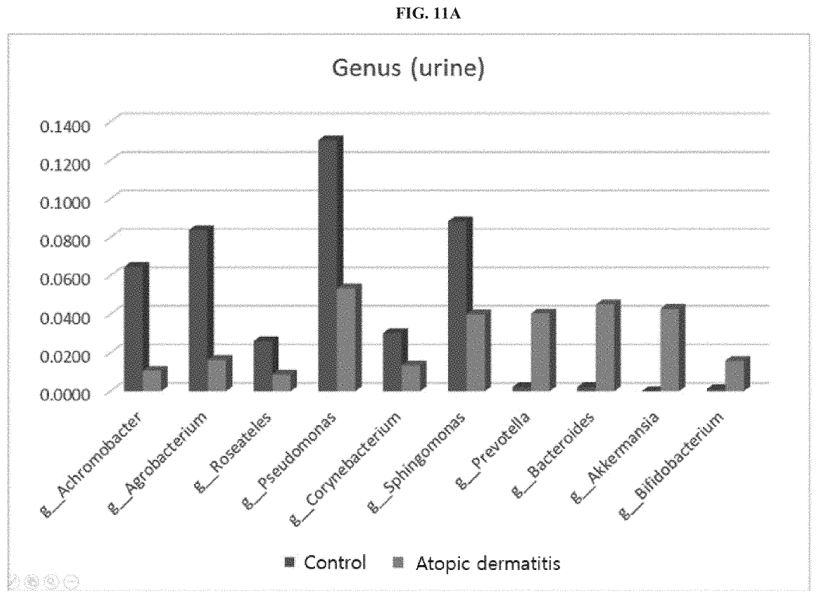

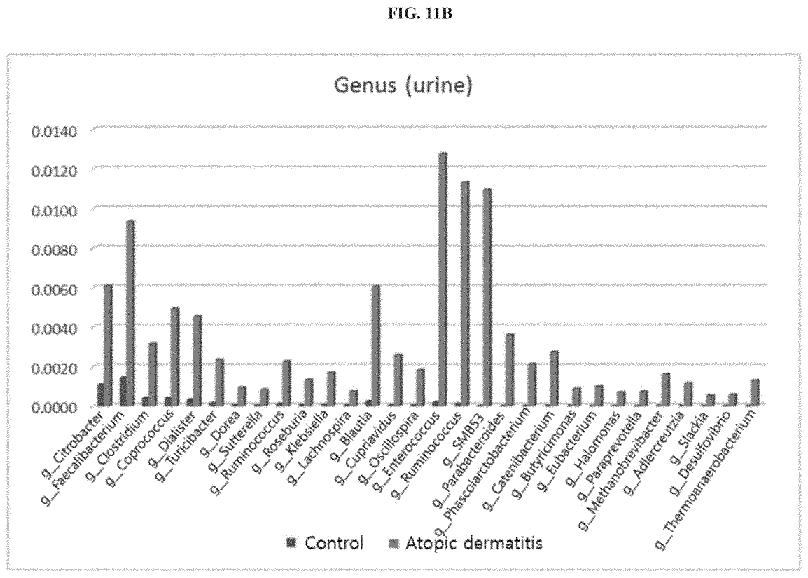

As a result of analyzing bacteria-derived EVs in urine at a genus level, a diagnostic model developed using bacteria belonging to the genus Achromobacter, the genus Agrobacterium, the genus Roseateles, the genus Pseudomonas, the genus Corynebacterium, the genus Sphingomonas, the genus Citrobacter, the genus Faecalibacterium, the genus Clostridium, the genus Coprococcus, the genus Dialister, the genus Bifidobacterium, the genus Turicibacter, the genus Dorea, the genus Sutterella, the genus Ruminococcus, the genus Prevotella, the genus Roseburia, the genus Bacteroides, the genus Klebsiella, the genus Lachnospira, the genus Blautia, the genus Cupriavidus, the genus Oscillospira, the genus Enterococcus, the genus Ruminococcus, the genus SMB53, the genus Akkermansia, the genus Parabacteroides, the genus Phascolarctobacterium, the genus Catenibacterium, the genus Butyricimonas, the genus Eubacterium, the genus Halomonas, the genus Paraprevotella, the genus Methanobrevibacter, the genus Adlercreutzia, the genus Slackia, the genus Desulfovibrio, and the genus Thermoanaerobacterium as a biomarker exhibited significant diagnostic performance for atopic dermatitis (see Table 11 and FIGS. 11A and 11B).

TABLE 11

| | | | Atopic | | t-test | | | Ac- | Sen- | Spe- |
|---|---|---|---|---|---|---|---|---|---|---|
| | Control | | dermatitis | | p- | | | cur- | sitiv- | cif- |
| Taxon | Mean | SD | Mean | SD | value | Ratio | AUC | acy | ity | city |
| g_Achromobacter | 0.0646 | 0.0684 | 0.0105 | 0.0217 | 0.0000 | 0.16 | 0.85 | 0.76 | 0.58 | 0.87 |
| g_Agrobacterium | 0.0837 | 0.0660 | 0.0162 | 0.0329 | 0.0000 | 0.19 | 0.88 | 0.79 | 0.63 | 0.89 |
| g_Roseateles | 0.0260 | 0.0312 | 0.0087 | 0.0169 | 0.0002 | 0.33 | 0.78 | 0.65 | 0.27 | 0.88 |
| g_Pseudomonas | 0.1304 | 0.0896 | 0.0535 | 0.0545 | 0.0000 | 0.41 | 0.79 | 0.68 | 0.46 | 0.82 |
| g_Corynebacterium | 0.0302 | 0.0234 | 0.0134 | 0.0164 | 0.0000 | 0.44 | 0.75 | 0.71 | 0.42 | 0.88 |
| g_Sphingomonas | 0.0882 | 0.0470 | 0.0400 | 0.0565 | 0.0000 | 0.45 | 0.76 | 0.69 | 0.47 | 0.82 |
| g_Citrobacter | 0.0011 | 0.0051 | 0.0061 | 0.0090 | 0.0000 | 5.57 | 0.77 | 0.60 | 0.05 | 0.93 |
| g_Faecalibacterium | 0.0014 | 0.0108 | 0.0093 | 0.0086 | 0.0000 | 6.51 | 0.82 | 0.76 | 0.98 | 0.62 |
| g_Clostridium | 0.0004 | 0.0021 | 0.0032 | 0.0040 | 0.0000 | 7.54 | 0.76 | 0.73 | 0.90 | 0.62 |
| g_Coprococcus | 0.0004 | 0.0030 | 0.0049 | 0.0064 | 0.0000 | 12.56 | 0.80 | 0.75 | 0.98 | 0.60 |
| g_Dialister | 0.0003 | 0.0018 | 0.0045 | 0.0053 | 0.0000 | 13.57 | 0.79 | 0.73 | 0.97 | 0.58 |
| g_Bifidobacterium | 0.0010 | 0.0049 | 0.0157 | 0.0136 | 0.0000 | 15.13 | 0.82 | 0.75 | 0.93 | 0.64 |
| g_Turicibacter | 0.0001 | 0.0009 | 0.0023 | 0.0029 | 0.0000 | 16.28 | 0.79 | 0.72 | 0.97 | 0.57 |
| g_Dorea | 0.0001 | 0.0004 | 0.0009 | 0.0011 | 0.0000 | 16.88 | 0.76 | 0.70 | 0.97 | 0.54 |
| g_Sutterella | 0.0000 | 0.0003 | 0.0008 | 0.0011 | 0.0000 | 18.18 | 0.77 | 0.69 | 0.69 | 0.69 |
| g_Ruminococcus | 0.0001 | 0.0009 | 0.0023 | 0.0024 | 0.0000 | 18.45 | 0.79 | 0.74 | 0.98 | 0.59 |
| g_Prevotella | 0.0021 | 0.0079 | 0.0404 | 0.0389 | 0.0000 | 19.14 | 0.84 | 0.75 | 0.90 | 0.66 |
| g_Roseburia | 0.0001 | 0.0005 | 0.0013 | 0.0015 | 0.0000 | 20.71 | 0.77 | 0.72 | 0.98 | 0.56 |
| g_Bacteroides | 0.0022 | 0.0163 | 0.0451 | 0.0591 | 0.0000 | 20.94 | 0.83 | 0.77 | 0.98 | 0.64 |
| g_Klebsiella | 0.0001 | 0.0004 | 0.0017 | 0.0019 | 0.0000 | 21.92 | 0.79 | 0.72 | 0.97 | 0.57 |
| g_Lachnospira | 0.0000 | 0.0003 | 0.0008 | 0.0010 | 0.0000 | 23.08 | 0.74 | 0.75 | 0.95 | 0.62 |
| g_Blautia | 0.0002 | 0.0017 | 0.0061 | 0.0090 | 0.0000 | 24.63 | 0.80 | 0.73 | 0.97 | 0.59 |
| g_Cupriavidus | 0.0001 | 0.0004 | 0.0026 | 0.0032 | 0.0000 | 50.03 | 0.79 | 0.75 | 0.98 | 0.60 |
| g_Oscillospira | 0.0000 | 0.0002 | 0.0018 | 0.0022 | 0.0000 | 65.19 | 0.81 | 0.73 | 0.98 | 0.57 |
| g_Enterococcus | 0.0002 | 0.0012 | 0.0128 | 0.0409 | 0.0032 | 69.57 | 0.85 | 0.78 | 0.98 | 0.65 |
| g_Ruminococcus | 0.0001 | 0.0009 | 0.0113 | 0.0123 | 0.0000 | 96.23 | 0.83 | 0.76 | 0.98 | 0.63 |
| g_SMB53 | 0.0000 | 0.0000 | 0.0109 | 0.0127 | 0.0000 | 6958.76 | 0.82 | 0.76 | 0.98 | 0.63 |
| g_Akkermansia | 0.0000 | 0.0000 | 0.0428 | 0.0479 | 0.0000 | | 0.83 | 0.78 | 1.00 | 0.64 |
| g_Parabacteroides | 0.0000 | 0.0000 | 0.0036 | 0.0043 | 0.0000 | | 0.82 | 0.75 | 1.00 | 0.60 |
| g_Phascolarcto- | 0.0000 | 0.0000 | 0.0021 | 0.0026 | 0.0000 | | 0.81 | 0.75 | 1.00 | 0.60 |
| bacterium | | | | | | | | | | |
| g_Catenibacterium | 0.0000 | 0.0000 | 0.0027 | 0.0035 | 0.0000 | | 0.79 | 0.74 | 1.00 | 0.58 |
| g_Butyricimonas | 0.0000 | 0.0000 | 0.0009 | 0.0015 | 0.0000 | | 0.78 | 0.70 | 1.00 | 0.52 |
| g_Eubacterium | 0.0000 | 0.0000 | 0.0010 | 0.0012 | 0.0000 | | 0.78 | 0.73 | 1.00 | 0.56 |
| g_Halomonas | 0.0000 | 0.0000 | 0.0007 | 0.0015 | 0.0008 | | 0.77 | 0.66 | 0.58 | 0.74 |
| g_Paraprevotella | 0.0000 | 0.0000 | 0.0007 | 0.0014 | 0.0001 | | 0.77 | 0.71 | 1.00 | 0.53 |
| g_Methanobrevi- | 0.0000 | 0.0000 | 0.0016 | 0.0027 | 0.0000 | | 0.77 | 0.68 | 1.00 | 0.49 |
| bacter | | | | | | | | | | |
| g_Adlercreutzia | 0.0000 | 0.0000 | 0.0011 | 0.0017 | 0.0000 | | 0.75 | 0.71 | 1.00 | 0.53 |
| g_Slackia | 0.0000 | 0.0000 | 0.0005 | 0.0009 | 0.0000 | | 0.75 | 0.66 | 1.00 | 0.46 |
| g_Desulfovibrio | 0.0000 | 0.0000 | 0.0006 | 0.0015 | 0.0033 | | 0.72 | 0.73 | 0.95 | 0.59 |
| g_Thermoanaero- | 0.0000 | 0.0000 | 0.0013 | 0.0022 | 0.0000 | | 0.72 | 0.68 | 0.54 | 0.77 |
| bacterium | | | | | | | | | | |

The above description of the present invention is provided only for illustrative purposes, and it will be understood by one of ordinary skill in the art to which the present invention pertains that the invention may be embodied in various modified forms without departing from the spirit or essential characteristics thereof. Thus, the embodiments described herein should be considered in an illustrative sense only and not for the purpose of limitation.

### INDUSTRIAL APPLICABILITY

A method of providing information on the diagnosis of atopic dermatitis through bacterial metagenomic analysis according to the present invention can be used to predict the risk of the onset of atopic dermatitis and diagnose atopic dermatitis by analyzing an increase or decrease in content of specific bacteria-derived extracellular vesicles using normal individual and subject-derived samples.

What is claimed is:

1. A method of providing information for diagnosing atopic dermatitis, the method comprising:
(a) extracting DNAs from extracellular vesicles isolated from normal individual and subject samples;
(b) performing polymerase chain reaction (PCR) on the extracted DNA using a pair of primers comprising SEQ ID NO: 1 and SEQ ID NO: 2; and
(c) comparing an increase or decrease in content of bacteria-derived extracellular vesicles of the subject-derived sample with that of a normal individual-derived sample through sequencing of a product of the PCR.

2. The method of claim 1, wherein process (c) comprises comparing an increase or decrease in content of extracellular vesicles derived from one or more bacteria selected from the group consisting of the phylum Cyanobacteria, the phylum Fusobacteria, the phylum Verrucomicrobia, the phylum Euryarchaeota, the phylum Firmicutes, the phylum Bacteroidetes and the phylum Tenericutes.

3. The method of claim 1, wherein process (c) comprises comparing an increase or decrease in content of extracellular vesicles derived from one or more bacteria selected from the group consisting of the class Chloroplast, the class Saprospirae, the class Flavobacteriia, the class Alphaproteobacteria, the class Fusobacteriia, the class Bacilli, the class Verrucomicrobiae, the class Methanobacteria, the class Betaproteobacteria, the class Coriobacteriia, the class Clostridia, the class Bacteroidia, the class Erysipelotrichi, the class Mollicutes, and the class Pedosphaerae.

4. The method of claim 1, wherein process (c) comprises comparing an increase or decrease in content of extracellular vesicles derived from one or more bacteria selected from the group consisting of the order Stramenopiles, the order Pseudomonadales, the order Neisseriales, the order Streptophyta, the order Rhizobiales, the order Saprospirales, the order Sphingomonadales, the order Flavobacteriales, the order Caulobacterales, the order Gemellales, the order Pasteurellales, the order Fusobacteriales, the order Rhodobacterales, the order Bacillales, the order Oceanospirillales, the order Enterobacteriales, the order Bifidobacteriales, the order Verrucomicrobiales, the order Methanobacteriales, the order Desulfovibrionales, the order MLE1-12, the order Burkholderiales, the order Coriobacteriales, the order Clostridiales, the order Bacteroidales, the order Erysipelotrichales, the order Turicibacterales, the order RF39, and the order Pedosphaerales.

5. The method of claim 1, wherein process (c) comprises comparing an increase or decrease in content of extracellular vesicles derived from one or more bacteria selected from the group consisting of the family Exiguobacteraceae, the family Moraxellaceae, the family Bradyrhizobiaceae, the family Rhizobiaceae, the family Flavobacteriaceae, the family Campylobacteraceae, the family Neisseriaceae, the family Pseudomonadaceae, the family Sphingomonadaceae, the family Chitinophagaceae, the family Carnobacteriaceae, the family Caulobacteraceae, the family Weeksellaceae, the family Methylobacteriaceae, the family Gemellaceae, the family Dermabacteraceae, the family Propionibacteriaceae, the family Pasteurellaceae, the family Leptotrichiaceae, the family Oxalobacteraceae, the family Fusobacteriaceae, the family Aerococcaceae, the family Rhodobacteraceae, the family Intrasporangiaceae, the family Paraprevotellaceae, the family Porphyromonadaceae, the family Staphylococcaceae, the family Corynebacteriaceae, the family Tissierellaceae, the family Micrococcaceae, the family Actinomycetaceae, the family Planococcaceae, the family Alcaligenaceae, the family mitochondria, the family Comamonadaceae, the family Veillonellaceae, the family Bifidobacteriaceae, the family Coriobacteriaceae, the family Clostridiaceae, the family Erysipelotrichaceae, the family Turicibacteraceae, the family Lachnospiraceae, the family Prevotellaceae, the family Rikenellaceae, the family Bacteroidaceae, the family Enterococcaceae, the family Ruminococcaceae, the family Desulfovibrionaceae, the family Verrucomicrobiaceae, the family Odoribacteraceae, the family Christensenellaceae, the family Methanobacteriaceae, the family Koribacteraceae, and the family Streptomycetaceae.

6. The method of claim 1, wherein process (c) comprises comparing an increase or decrease in content of extracellular vesicles derived from one or more bacteria selected from the group consisting of the genus Exiguobacterium, the genus Acinetobacter, the genus Capnocytophaga, the genus Proteus, the genus Neisseria, the genus Sphingomonas, the genus Pseudomonas, the genus Aggregatibacter, the genus Leptotrichia, the genus Granulicatella, the genus Prevotella, the genus Chryseobacterium, the genus Porphyromonas, the genus Haemophilus, the genus Brachybacterium, the genus Propionibacterium, the genus Eubacterium, the genus Fusobacterium, the genus Enhydrobacter, the genus Paracoccus, the genus Parabacteroides, the genus Staphylococcus, the genus Corynebacterium, the genus Rothia, the genus Actinomyces, the genus Dialister, the genus Faecalibacterium, the genus Dorea, the genus Ruminococcus, the genus Halomonas, the genus Sutterella, the genus Bacteroides, the genus Veillonella, the genus Rhodococcus, the genus Butyricimonas, the genus Akkermansia, the genus Bifidobacterium, the genus Atopobium, the genus Citrobacter, the genus Klebsiella, the genus Enterobacter, the genus Chromohalobacter, the genus Cupriavidus, the genus Methanobrevibacter, the genus Phascolarctobacterium, the genus Odoribacter, the genus Pyramidobacter, the genus Bilophila, the genus Desulfovibrio, the genus Acidaminococcus, the genus Achromobacter, the genus Agrobacterium, the genus Roseateles, the genus Clostridium, the genus Coprococcus, the genus Turicibacter, the genus Roseburia, the genus Lachnospira, the genus Blautia, the genus Oscillospira, the genus Enterococcus, the genus SMB53, the genus Catenibacterium, the genus Paraprevotella, the genus Adlercreutzia, the genus Slackia, and the genus Thermoanaerobacterium.

7. The method of claim 1, wherein h normal individual and subject sample is blood or urine.

8. (canceled)

9. The method of claim 1, wherein process (c) comprises comparing an increase or decrease in content of extracellular vesicles derived from one or more bacteria selected from the group consisting of the phylum Cyanobacteria, the phylum Fusobacteria, the phylum Verrucomicrobia and the phylum Euryarchaeota, which are isolated from normal individual and subject-derived blood samples, and the phylum Cyanobacteria, the phylum Firmicutes, the phylum Bacteroidetes, the phylum Verrucomicrobia, the phylum Euryarchaeota and the phylum Tenericutes, which are isolated from normal individual and subject-derived urine samples;
extracellular vesicles derived from one or more bacteria selected from the group consisting of the class Chloroplast, the class Saprospirae, the class Flavobacteriia, the class Alphaproteobacteria, the class Fusobacteriia, the class Bacilli, the class Verrucomicrobiae and the class Methanobacteria, which are isolated from normal individual and subject-derived blood samples, and the class Chloroplast, the class Betaproteobacteria, the class Coriobacteriia, the class Clostridia, the class Bacteroidia, the class Erysipelotrichi, the class Verrucomicrobiae, the class Methanobacteria, the class Mollicutes and the class Pedosphaerae, which are isolated from normal individual and subject-derived urine samples;
extracellular vesicles derived from one or more bacteria selected from the group consisting of the order Stramenopiles, the order Pseudomonadales, the order Neisseriales, the order Streptophyta, the order Rhizobiales, the order Saprospirales, the order Sphingomonadales, the order Flavobacteriales, the order Caulobacterales, the order Gemellales, the order Pasteurellales, the order Fusobacteriales, the order Rhodobacterales, the order Bacillales, the order Lactobacillales, the order Oceanospirillales, the order Enterobacteriales, the order Bifidobacteriales, the order Verrucomicrobiales, the order Methanobacteriales and the order Desulfovibrionales, which are isolated from normal individual and subject-derived blood samples, and MLE1-12, the order Burkholderiales, the order Streptophyta, the order Pseudomonadales, the order Sphingomonadales, the order Bifidobacteriales, the order Coriobacteriales, the order Clostridiales, the order Bacteroidales, the order Erysipelotrichales, the order Turicibacterales, the order Desulfovibrionales, the order Verrucomicrobiales, the order Methanobacteriales, the order RF39 and the order Pedosphaerales, which are isolated from normal individual and subject-derived urine samples;
extracellular vesicles derived from one or more bacteria selected from the group consisting of the family Exiguobacteraceae, the family Moraxellaceae, the family Bradyrhizobiaceae, the family Rhizobiaceae, the family Flavobacteriaceae, the family Campylobacteraceae, the family Neisseriaceae, the family Pseudomonadaceae, the family Sphingomonadaceae, the family Chitinophagaceae, the family Carnobacteriaceae, the family Caulobacteraceae, the family Weeksellaceae, the family Methylobacteriaceae, the family Gemellaceae, the family Dermabacteraceae, the family Propionibacteriaceae, the family Pasteurellaceae, the family Leptotrichiaceae, the family Oxalobacteraceae, the family Fusobacteriaceae, the family Aerococcaceae, the family Rhodobacteraceae, the family Intrasporangiaceae, the family Paraprevotellaceae, the family Porphyromonadaceae, the family Staphylococcaceae, the family Corynebacteriaceae, the family Tissierellaceae, the family Micrococcaceae, the family Actinomycetaceae, the family Planococcaceae, the family Comamonadaceae, the family Halomonadaceae, the family Clostridiaceae, the family Alcaligenaceae, the family Enterobacteriaceae, the family Bacteroidaceae, the family Peptostreptococcaceae, the family Nocardiaceae, the family Bifidobacteriaceae, the family Verrucomicrobiaceae, the family Shewanellaceae, the family Barnesiellaceae, the family Odoribacteraceae, the family Methanobacteriaceae, the family Rikenellaceae, the family Desulfovibrionaceae, and the family Dethiosulfovibrionaceae, which are isolated from normal individual and subject-derived blood samples, and the family Alcaligenaceae, the faintly Rhizobiaceae, the family mitochondria, the family Pseudomonadaceae, the family Corynebacteriaceae, the family Comamonadaceae, the family Rhodobacteraceae, the family Sphingomonadaceae, the family Veillonellaceae, the family Bifidobacteriaceae, the family Coriobacteriaceae, the family Planococcaceae, the family Paraprevotellaceae, the family Clostridiaceae, the family Erysipelotrichaceae, the family Turicibacteraceae, the family Lachnospiraceae, the family Prevotellaceae, the family Rikenellaceae, the family Bacteroidaceae, the family Enterococcaceae, the family Ruminococcaceae, the family Desulfovibrionaceae, the family Verrucomicrobiaceae, the family Odoribacteraceae, the family Christensenellaceae, the family Methanobacteriaceae, the family Koribacteraceae and the family Streptomycetaceae, which are isolated from normal individual and subject-derived urine samples; or
extracellular vesicles derived from one or more bacteria selected from the group consisting of the genus Exiguobacterium, the genus Acinetobacter, the genus Capnocytophaga, the genus Proteus, the genus Neisseria, the genus Sphingomonas, the genus Pseudomonas, the genus Aggregatibacter, the genus Leptotrichia, the genus Granulicatella, the genus Prevotella, the genus Chryseobacterium, the genus Porphyromonas, the genus Haemophilus, the genus Brachybacterium, the genus Propionibacterium, the genus Eubacterium, the genus Fusobacterium, the genus Enhydrobacter, the genus Paracoccus, the genus Parabacteroides, the genus Staphylococcus, the genus Corynebacterium, the genus Rothia, the genus Actinomyces, the genus Dialister, the genus Faecalibacterium, the genus Dorea, the genus Ruminococcus, the genus Halomonas, the genus Sutterella, the genus Bacteroides, the genus Veillonella, the genus Rhodococcus, the genus Butyricimonas, the genus Akkermansia, the genus Bifidobacterium, the genus Atopobium, the genus Citrobacter, the genus Klebsiella, the genus Enterobacter, the genus Chromohalobacter, the genus Cupriavidus, the genus Methanobrevibacter, the genus Phascolarctobacterium, the genus Odoribacter, the genus Pyramidobacter, the genus Bilophila, the genus Desulfovibrio, and the genus Acidaminococcus, which are isolated from normal individual and subject-derived blood samples, and the genus Achromobacter, the genus Agrobacterium, the genus Roseateles, the genus Pseudomonas, the genus Corynebacterium, the genus Sphingomonas, the genus Citrobacter, the genus Faecalibacterium, the genus Clostridium, the genus Coprococcus, the genus Dialister, the genus Bifidobacterium, the genus Turicibacter, the genus Dorea, the genus Sutterella, the genus Ruminococcus, the genus Prevotella, the genus Roseburia, the genus Bacteroides, the genus Klebsiella, the genus Lachnospira, the genus Blautia, the genus Cupriavidus, the genus Oscillospira, the genus Enterococcus, the genus SMB53, the genus Akkermansia, the genus Parabacteroides, the genus Phascolarctobacterium, the genus Catenibacterium, the genus Butyricimonas, the genus Eubacterium, the genus Halomonas, the genus Paraprevotella, the genus Methanobrevibacter, the genus Adlercreutzia, the genus Slackia, the genus Desulfovibrio and the genus Thermoanaerobacterium, which are isolated from normal individual and subject-derived urine samples.

10. The method of claim 9, wherein process (c), in comparison with the normal individual-derived sample, an increase in the content of the following is diagnosed as atopic dermatitis:
extracellular vesicles derived from one or more bacteria selected from the group consisting of the phylum Verrucomicrobia, and the phylum Euryarchaeota, which are isolated from subject-derived blood samples, and the phylum Firmicutes, the phylum Bacteroidetes, the phylum Verrucomicrobia, the phylum Euryarchaeota, and the phylum Tenericutes, which are isolated from subject-derived urine samples,
extracellular vesicles derived from one or more bacteria selected from the group consisting of the class Bacilli, the class Verrucomicrobiae, and the class Methanobacteria, which are isolated from subject-derived blood samples, and the class Coriobacteriia, the class Clostridia, the class Bacteroidia, the class Erysipelotrichi, the class Verrucomicrobiae, the class Methanobacteria, the class Mollicutes, and the class Pedosphaerae, which are isolated from subject-derived urine samples,
extracellular vesicles derived from one or more bacteria selected from the group consisting of the order Lactobacillales, the order Oceanospirillales, the order Enterobacteriales, the order Bifidobacteriales, the order Verrucomicrobiales, the order Methanobacteriales, and the order Desulfovibrionales, which are isolated from subject-derived blood samples, and the order Bifidobacteriales, the order Coriobacteriales, the order Clostridiales, the order Bacteroidales, the order Erysipelotrichales, the order Turicibacterales, the order Desulfovibrionales, the order Verrucomicrobiales, the order Methanobacteriales, the order RF39, and the order Pedosphaerales, which are isolated from subject-derived urine samples,
extracellular vesicles derived from one or more bacteria selected from e group consisting of the family Comamonadaceae, the family Halomonadaceae, the family Clostridiaceae, the family Alcaligenaceae, the family Enterobacteriaceae, the family Bacteroidaceae, the family Peptostreptococcaceae, the family Nocardiaceae, the family Bifidobacteriaceae, the family Verrucomicrobiaceae, the family Shewanellaceae, the family Barnesiellaceae, the family Odoribacteraceae, the family Methanobacteriaceae, the family Rikenellaceae, the family Desulfovibrionaceae, and the family Dethiosulfovibrionaceae, which are isolated from subject-derived blood samples, and the family Veillonellaceae, the family Bifidobacteriaceae, the family Coriobacteriaceae, the family Planococcaceae, the family Paraprevotellaceae, the family Clostridiaceae, the family Erysipelotrichaceae, the family Turicibacteraceae, the family Lachnospiraceae, the family Prevotellaceae, the family Rikenellaceae, the family Bacteroidaceae, the family Enterococcaceae, the family Ruminococcaceae, the family Desulfovibrionaceae, the family Verrucomicrobiaceae, the family Odoribacteraceae, the family Christensenellaceae, the family Methanobacteriaceae, the family Koribacteraceae, and the family Streptomycetaceae, which are isolated from subject-derived urine samples, or
extracellular vesicles derived from one or more bacteria selected from the group consisting of the genus Ruminococcus, the genus Halomonas, the genus Sutterella, the genus Bacteroides, the genus Veillonella, the genus Rhodococcus, the genus Butyricimonas, the genus Akkermansia, the genus Bifidobacterium, the genus Atopobium, the genus Citrobacter, the genus Klebsiella, the genus Enterobacter, the genus Chromohalobacter, the genus Cupriavidus, the genus Methanobrevibacter, the genus Phascolarctobacterium, the genus Odoribacter, the genus Pyramidobacter, the genus Bilophila, the genus Desulfovibrio, and the genus Acidaminococcus, which are isolated from subject-derived blood samples, and the genus Citrobacter, the genus Faecalibacterium, the genus Clostridium, the genus Coprococcus, the genus Dialister, the genus Bifidobacterium, the genus Turicibacter, the genus Dorea, the genus Sutterella, the genus Ruminococcus, the genus Prevotella, the genus Roseburia, the genus Bacteroides, the genus Klebsiella, the genus Lachnospira, the genus Blautia, the genus Cupriavidus, the genus Oscillospira, the genus Enterococcus, the genus Ruminococcus, the genus SMB53, the genus Akkermansia, the genus Parabacteroides, the genus Phascolarctobacterium, the genus Catenibacterium, the genus Butyricimonas, the genus Eubacterium, the genus Halomonas, the genus Paraprevotella, the genus Methanobrevibacter, the genus Adlercreutzia, the genus Slackia, the genus Desulfovibrio, and the genus Thermoanaerobacterium, which are isolated from subject-derived urine samples.

11. The method of claim 9, wherein process (c), in comparison with the normal individual-derived sample, a decrease in the content of the following is diagnosed as atopic dermatitis:
extracellular vesicles derived from one or more bacteria selected from the group consisting of the phylum Cyanobacteria, and the phylum Fusobacteria, which are isolated from subject-derived blood samples, and the phylum Cyanobacteria, which are isolated from subject-derived urine samples,
extracellular vesicles derived from one or more bacteria selected from the group consisting of the class Chloroplast, the class Saprospirae, the class Flavobacteriia, the class Alphaproteobacteria, and the class Fusobacteriia, which are isolated from subject-derived blood samples, and the class Chloroplast, and the class Betaproteobacteria, which are isolated from subject-derived urine samples,
extracellular vesicles derived from one or more bacteria selected from the group consisting of the order Stramenopiles, the order Pseudomonadales, the order Neisseriales, the order Streptophyta, the order Rhizobiales, the order Saprospirales, the order Sphingomonadales, the order Flavobacteriales, the order Caulobacterales, the order Gemellales, the order Pasteurellales, the order Fusobacteriales, the order Rhodobacterales, and the order Bacillales, which are isolated from subject-derived blood samples, and the order MLE1-12, the order Burkholderiales, the order Streptophyta, the order Pseudomonadales, and the order Sphingomonadales, which are isolated from subject-derived urine samples,
extracellular vesicles derived from one or more bacteria selected from the group consisting of the family Exiguobacteraceae, the family Moraxellaceae, the family Bradyrhizobiaceae, the family Rhizobiaceae, the family Flavobacteriaceae, the family Campylobacteraceae, the family Neisseriaceae, the family Pseudomonadaceae, the family Sphingomonadaceae, the family Chitinophagaceae, the family Carnobacteriaceae, the family Caulobacteraceae, the family Weeksellaceae, the family Methylobacteriaceae, the family Gemellaceae, the family Dermabacteraceae, the family Propionibacteriaceae, the family Pasteurellaceae, the family Leptotrichiaceae, the family Oxalobacteraceae, the family Fusobacteriaceae, the family Aerococcaceae, the family Rhodobacteraceae, the family Intrasporangiaceae, the family Paraprevotellaceae, the family Porphyromonadaceae, the family Staphylococcaceae, the family Corynebacteriaceae, the family Tissierellaceae, the family Micrococcaceae, the family Actinomycetaceae,and the family Planococcaceae, which are isolated from subject-derived blood samples, and the family Alcaligenaceae, the family Rhizobiaceae, the family mitochondria, the family Pseudomonadaceae, the family. Corynebacteriaceae, the family Comamonadaceae, the family Rhodobacteraceae, and the family Sphingomonadaceae, which are isolated from subject-derived urine samples, or
extracellular vesicles derived from one or more bacteria selected from the group consisting of the genus Exiguobacterium, the genus Acinetobacter, the genus Capnocytophaga, the genus Proteus, the genus Neisseria, the genus Sphingomonas, the genus Pseudomonas, the genus Aggregatibacter, the genus Leptotrichia, the genus Granulicatella, the genus Prevotella, the genus Chryseobacterium, the genus Porphyromonas, the genus Haemophilus, the genus Brachybacterium, the genus Propionibacterium, the genus Eubacterium, the genus Fusobacterium, the genus Enhydrobacter, the genus Paracoccus, the genus Parabacteroides, the genus Staphylococcus, the genus Corynebacterium, the genus Rothia, the genus Actinomyces, the genus Dialister, the genus Faecalibacterium, and the genus Dorea, which are isolated from subject-derived blood samples, and the genus Achromobacter, the genus Agrobacterium, the genus Roseateles, the genus Pseudomonas, the genus Corynebacterium, and the genus Sphingomonas, which are isolated from subject-derived urine samples.

12. A method of diagnosing atopic dermatitis, the method comprising:
(a) extracting DNAs from extracellular vesicles isolated from normal individual and subject samples;
(b) performing polymerase chain reaction (PCR) on the extracted DNA using a pair of primers comprising SEQ ID NO: 1 and SEQ ID NO: 2; and
(c) comparing an increase or decrease in content of bacteria-derived extracellular vesicles of the subject-derived sample with that of a normal individual-derived sample through sequencing of a product of the PCR.

13. The method of claim 12, wherein process (c) comprises comparing an increase or decrease in content of extracellular vesicles derived from one or more bacteria selected from the group consisting of the phylum Cyanobacteria, the phylum Fusobacteria, the phylum Verrucomicrobia, the phylum Euryarchaeota, the phylum Firmicutes, the phylum Bacteroidetes and the phylum Tenericutes.

14. The method of claim 12, wherein process (c) comprises comparing an increase or decrease in content of extracellular vesicles derived from one or more bacteria selected from the group consisting of the class Chloroplast, the class Saprospirae, the class Flavobacteriia, the class Alphaproteobacteria, the class Fusobacteriia, the class Bacilli, the class Verrucomicrobiae, the class Methanobacteria, the class Betaproteobacteria, the class Coriobacteriia, the class Clostridia, the class Bacteroidia, the class Erysipelotrichi, the class Mollicutes, and the class Pedosphaerae.

15. The method of claim 12, wherein process (c) comprises comparing an increase or decrease in content of extracellular vesicles derived from one or more bacteria selected from the group consisting of the order Stramenopiles, the order Pseudomonadales, the order Neisseriales, the order Streptophyta, the order Rhizobiales, the order Saprospirales, the order Sphingomonadales, the order Flavobacteriales, the order Caulobacterales, the order Gemellales, the order Pasteurellales, the order Fusobacteriales, the order Rhodobacterales, the order Bacillales, the order Oceanospirillales, the order Enterobacteriales, the order Bifidobacteriales, the order Verrucomicrobiales, the order Methanobacteriales, the order Desulfovibrionales, the order MILE1-12, the order Burkholderiales, the order Coriobacteriales, the order Clostridiales, the order Bacteroidales, the order Erysipelotrichales, the order Turicibacterales, the order RF39, and the order Pedosphaerales.

16. The method of claim 12, wherein process (c) comprises comparing an increase or decrease in content of extracellular vesicles derived from one or more bacteria selected froth the group consisting of the family Exiguobacteraceae, the family Moraxellaceae, the family Bradyrhizobiaceae, the family Rhizobiaceae, the family Flavobacteriaceae, the family Campylobacteraceae, the family Neisseriaceae, the family Pseudomonadaceae, the family Sphingomonadaceae, the family Chitinophagaceae, the family Carnobacteriaceae, the family Caulobacteraceae, the family Weeksellaceae, the family Methylobacteriaceae, the family Gemellaceae, the family Dermabacteraceae, the family Propionibacteriaceae, the family Pasteurellaceae, the family Leptotrichiaceae, the family Oxalobacteraceae, the family Fusobacteriaceae, the family Aerococcaceae, the family Rhodobacteraceae, the family Intrasporangiaceae, the family Paraprevotellaceae, the family Porphyromonadaceae, the family Staphylococcaceae, the family Corynebacteriaceae, the family Tissierellaceae, the family Micrococcaceae, the family Actinomycetaceae, the family Planococcaceae, the family Alcaligenaceae, the family mitochondria, the family Comamonadaceae, the family Veillonellaceae, the family Bifidobacteriaceae, the family Coriobacteriaceae, the family Clostridiaceae, the family Erysipelotrichaceae, the family Turicibacteraceae, the family Lachnospiraceae, the family Prevotellaceae, the family Rikenellaceae, the family Bacteroidaceae, the family Enterococcaceae, the family Ruminococcaceae, the family Desulfovibrionaceae, the family Verrucomicrobiaceae, the family Odoribacteraceae, the family Christensenellaceae, the family Methanobacteriaceae, the family Koribacteraceae, and the family Streptomycetaceae.

17. The method of claim 12, wherein process (c) comprises comparing an increase or decrease in content of extracellular vesicles derived from one or more bacteria selected from the group consisting of the genus Exiguobacterium, the genus Acinetobacter, the genus Capnocytophaga, the genus Proteus, the genus Neisseria, the genus Sphingomonas, the genus Pseudomonas, the genus Aggregatibacter, the genus Leptotrichia, the genus Granulicatella, the genus Prevotella, the genus Chryseobacterium, the genus Porphyromonas, the genus Haemophilus, the genus Brachybacterium, the genus Propionibacterium, the genus Eubacterium, the genus Fusobacterium, the genus Enhydrobacter, the genus Paracoccus, the genus Parabacteroides, the genus Staphylococcus, the genus Corynebacterium, the genus Rothia, the genus Actinomyces, the genus Dialister, the genus Faecalibacterium, the genus Dorea, the genus Ruminococcus, the genus Halomonas, the genus Sutterella, the genus Bacteroides, the genus Veillonella, the genus Rhodococcus, the genus Butyricimonas, the genus Akkermansia, the genus Bifidobacterium, the genus Atopobium, the genus Citrobacter, the genus Klebsiella, the genus Enterobacter, the genus Chromohalobacter, the genus Cupriavidus, the genus Methanobrevibacter, the genus Phascolarctobacterium, the genus Odoribacter, the genus Pyramidobacter, the genus Bilophila, the genus Desulfovibrio, the genus Acidaminococcus, the genus Achromobacter, the genus Agrobacterium, the genus Roseateles, the genus Clostridium, the genus Coprococcus, the genus Turicibacter, the genus Roseburia, the genus Lachnospira, the genus Blautia, the genus Oscillospira, the genus Enterococcus, the genus SMB53, the genus Catenibacterium, the genus Paraprevotella, the genus Adlercreutzia, the genus Slackia, and the genus Thermoanaerobacterium.

18. The method of claim 12, wherein the normal individual and subject sample is blood or urine.

19. (canceled)

20. The method of claim 12, wherein process (c) comprises comparing an increase or decrease in content of extracellular vesicles derived from one or more bacteria selected from the group consisting of the phylum Cyanobacteria, the phylum Fusobacteria, the phylum Verrucomicrobia and the phylum Euryarchaeota, which are isolated from normal individual and subject-derived blood samples, and the phylum Cyanobacteria, the phylum Firmicutes, the phylum Bacteroidetes, the phylum Verrucomicrobia, the phylum Euryarchaeota, and the phylum Tenericutes, which are isolated from normal individual and subject-derived urine samples;
extracellular vesicles derived from one or more bacteria selected from the group consisting of the class Chloroplast, the class Saprospirae, the class Flavobacteriia, the class Alphaproteobacteria, the class Fusobacteriia, the class Bacilli, the class Verrucomicrobiae and the class Methanobacteria, which are isolated from normal individual and subject-derived blood samples, and the class Chloroplast, the class Betaproteobacteria, the class Coriobacteriia, the class Clostridia, the class Bacteroidia, the class Erysipelotrichi, the class Verrucomicrobiae, the class Methanobacteria, the class Mollicutes and the class Pedosphaerae, which are isolated from normal individual and subject-derived urine samples;
extracellular vesicles derived from one or more bacteria selected from the group consisting of the order Stramenopiles, the order Pseudomonadales, the order Neisseriales, the order Streptophyta, the order Rhizobiales, the order Saprospirales, the order Sphingomonadales, the order Flavobacteriales, the order Caulobacterales, the order Gemellales, the order Pasteurellales, the order Fusobacteriales, the order Rhodobacterales, the order Bacillales, the order Lactobacillales, the order Oceanospirillales, the order Enterobacteriales, the order Bifidobacteriales, the order Verrucomicrobiales, the order Methanobacteriales and the order Desulfovibrionales, which are isolated from normal individual and subject-derived blood samples, and MLE1-12, the order Burkholderiales, the order Streptophyta, the order Pseudomonadales, the order Sphingomonadales, the order Bifidobacteriales, the order Coriobacteriales, the order Clostridiales, the order Bacteroidales, the order Erysipelotrichales, the order Turicibacterales, the order Desulfovibrionales, the order Verrucomicrobiales, the order Methanobacteriales, the order RF39 and the order Pedosphaerales, which are isolated from normal individual and subject-derived urine samples;
extracellular vesicles derived from one or more bacteria selected from the group consisting of the family Exiguobacteraceae, the family Moraxellaceae, the family Bradyrhizobiaceae, the family Rhizobiaceae, the family Flavobacteriaceae, the family Campylobacteraceae, the family Neisseriaceae, the family Pseudomonadaceae, the family Sphingomonadaceae, the family Chitinophagaceae, the family Carnobacteriaceae, the family Caulobacteraceae, the family Weeksellaceae, the family Methylobacteriaceae, the family Gemellaceae, the family Dermabacteraceae, the family Propionibacteriaceae, the family Pasteurellaceae, the family Leptotrichiaceae, the family Oxalobacteraceae, the family Fusobacteriaceae, the family Aerococcaceae, the family Rhodobacteraceae, the family Intrasporangiaceae, the family Paraprevotellaceae, the family Porphyromonadaceae, the family Staphylococcaceae, the family Corynebacteriaceae, the family Tissierellaceae, the family Micrococcaceae, the family Actinomycetaceae, the family Planococcaceae, the family Comamonadaceae, the family Halomonadaceae, the family Clostridiaceae, the family Alcaligenaceae, the family Enterobacteriaceae, the family Bacteroidaceae, the family Peptostreptococcaceae, the family Nocardiaceae, the family Bifidobacteriaceae, the family Verrucomicrobiaceae, the family Shewanellaceae, the family Barnesiellaceae, the family Odoribacteraceae, the family Methanobacteriaceae, the family Rikenellaceae, the family Desulfovibrionaceae, and the family Dethiosulfovibrionaceae, which are isolated from normal individual and subject-derived blood samples, and the family Alcaligenaceae, the family Rhizobiaceae, the family mitochondria, the family Pseudomonadaceae, the family Corynebacteriaceae, the family Comamonadaceae, the family Rhodobacteraceae, the family Sphingomonadaceae, the family Veillonellaceae, the family Bifidobacteriaceae, the family Coriobacteriaceae, the family Planococcaceae, the family Paraprevotellaceae, the family Clostridiaceae, the family Erysipelotrichaceae, the family Turicibacteraceae, the family Lachnospiraceae, the family Prevotellaceae, the family Rikenellaceae, the family Bacteroidaceae, the family Enterococcaceae, the family Ruminococcaceae, the family Desulfovibrionaceae, the family Verrucomicrobiaceae, the family Odoribacteraceae, the family Christensenellaceae, the family Methanobacteriaceae, the family Koribacteraceae and the family Streptomycetaceae, which are isolated from normal individual and subject-derived urine samples; or
extracellular vesicles derived from one or more bacteria selected from the group consisting of the genus Exiguobacterium, the genus Acinetobacter, the genus Capnocytophaga, the genus Proteus, the genus Neisseria, genus Sphingomonas, the genus Pseudomonas, the genus Aggregatibacter, the genus Leptotrichia, the genus Granulicatella, the genus Prevotella, the genus Chryseobacterium, the genus Porphyromonas, the genus Haemophilus, the genus Brachybacterium, the genus Propionibacterium, the genus Eubacterium, the genius Fusobacterium, the genus Enhydrobacter, the genus Paracoccus, the genus Parabacteroides, the genus Staphylococcus, the genus Corynebacterium, the genus Rothia, the genus Actinomyces, the genus Dialister, the genus Faecalibacterium, the genus Dorea, the genus Ruminococcus, the genus Halomonas, the genus Sutterella, the genus Bacteroides, the genus Veillonella, the genus Rhodococcus, the genus Butyricimonas, the genus Akkermansia, the genus Bifidobacterium, the genus Atopobium, the genus Citrobacter, the genus Klebsiella, the genus Enterobacter, the genus Chromohalobacter, the genus Cupriavidus, the genus Methanobrevibacter, the genus Phascolarctobacterium, the genus Odoribacter, the genus Pyramidobacter, the genus Bilophila, the genus Desulfovibrio and the genus Acidaminococcus, which are isolated from normal individual and subject-derived blood samples, and the genus Achromobacter, the genus Agrobacterium, the genus Roseateles, the genus Pseudomonas, the genus Corynebacterium, the genus Sphingomonas, the genus Citrobacter, the genus Faecalibacterium, the genus Clostridium, the genus Coprococcus, the genus Dialister, the genus Bifidobacterium, the genus Turicibacter, the genus Dorea, the genus Sutterella, the genus Ruminococcus, the genus Prevotella, the genus Roseburia, the genus Bacteroides, the genus Klebsiella, the genus Lachnospira, the genus Blautia, the genus Cupriavidus, the genus Oscillospira, the genus Enterococcus, the genus SMB53, the genus Akkermansia, the genus Parabacteroides, the genus Phascolarctobacterium, the genus Catenibacterium, the genus Butyricimonas, the genus Eubacterium, the genus Halomonas, the genus Paraprevotella, the genus Methanobrevibacter, the genus Adlercreutzia, the genus Slackia, the genus Desulfovibrio, and the genus Thermoanaerobacterium, which are isolated from normal individual and subject-derived urine samples.

21. The method of claim 20, wherein process (c), in comparison with the normal individual-derived sample, an increase in the content of the following is diagnosed as atopic dermatitis:
extracellular vesicles derived from one or more bacteria selected from the group consisting of the phylum Verrucomicrobia, and the phylum Euryarchaeota, which are isolated from subject-derived blood samples, and the phylum Firmicutes, the phylum Bacteroidetes, the phylum Verrucomicrobia, the phylum Euryarchaeota, and the phylum Tenericutes, which are isolated from subject-derived urine samples,
extracellular vesicles derived from one or more bacteria selected from the group consisting of the class Bacilli, the class Verrucomicrobiae, and the class Methanobacteria, which are isolated from subject-derived blood samples, and the class Coriobacteriia, the class Clostridia, the class Bacteroidia, the class Erysipelotrichi, the class Verrucomicrobiae, the class Methanobacteria, the class Mollicutes, and the class Pedosphaerae, which are isolated from subject-derived urine samples,
extracellular vesicles derived from one or more bacteria selected from the group consisting of the order Lactobacillales, the order Oceanospirillales, the order Enterobacteriales, the order Bifidobacteriales, the order Verrucomicrobiales, the order Methanobacteriales, and the order Desulfovibrionales, which are isolated from subject-derived blood samples, and the order Bifidobacteriales, the order Coriobacteriales, the order Clostridiales, the order Bacteroidales, the order Erysipelotrichales, the order Turicibacterales, the order Desulfovibrionales, the order Verrucomicrobiales, the order Methanobacteriales, the order RF39, and the order Pedosphaerales, which are isolated from subject-derived urine samples,
extracellular vesicles derived from one or more bacteria selected from the group consisting of the family Comamonadaceae, the family Halomonadaceae, the family Clostridiaceae, the family Alcaligenaceae, the family Enterobacteriaceae, the family Bacteroidaceae, the family Peptostreptococcaceae, the family Nocardiaceae, the family Bifidobacteriaceae, the family Verrucomicrobiaceae, the family Shewanellaceae, the family Barnesiellaceae, the family Odoribacteraceae, the family Methanobacteriaceae, the family Rikenellaceae, the family Desulfovibrionaceae, and the family Dethiosulfovibrionaceae, which are isolated from subject-derived blood samples, and the family Veillonellaceae, the family Bifidobacteriaceae, the family Coriobacteriaceae, the family Planococcaceae, the family Paraprevotellaceae, the family Clostridiaceae, the family Erysipelotrichaceae, the family Turicibacteraceae, the family Lachnospiraceae, the family Prevotellaceae, the family Rikenellaceae, the family Bacteroidaceae, the family Enterococcaceae, the family Ruminococcaceae, the family Desulfovibrionaceae, the family Verrucomicrobiaceae, the family Odoribacteraceae, the family Christensenellaceae, the family Methanobacteriaceae, the family Koribacteraceae, and the family Streptomycetaceae, which are isolated from subject-derived urine samples, or
extracellular vesicles derived from one or more bacteria selected from the group consisting of the genus Ruminococcus, the genus Halomonas, the genus Sutterella, the genus Bacteroides, the genus Veillonella, the genus Rhodococcus, the genus Butyricimonas, the genus Akkermansia, the genus Bifidobacterium, the genus Atopobium, the genus Citrobacter, the genus Klebsiella, the genus Enterobacter, the genus Chromohalobacter, the genus Cupriavidus, the genus Methanobrevibacter, the genus Phascolarctobacterium, the genus Odoribacter, the genus Pyramidobacter, the genus Bilophila, the genus Desulfovibrio, and the genus Acidaminococcus, which are isolated from subject-derived blood samples, and the genus Citrobacter, the genus Faecalibacterium, the genus Clostridium, the genus Coprococcus, the genus Dialister, the genus Bifidobacterium, the genus Turicibacter, the genus Dorea, the genus Sutterella, the genus Ruminococcus, the genus Prevotella, the genus Roseburia, the genus Bacteroides, the genus Klebsiella, the genus Lachnospira, the genus Blautia, the genus Cupriavidus, the genus Oscillospira, the genus Enterococcus, the genus Ruminococcus, the genus SMB53, the genus Akkermansia, the genus Parabacteroides, the genus Phascolarctobacterium, the genus Catenibacterium, the genus Butyricimonas, the genus Eubacterium, the genus Halomonas, the genus Paraprevotella, the genus Methanobrevibacter, the genus Adlercreutzia, the genus Slackia, the genus Desulfovibrio, and the genus Thermoanaerobacterium, which are isolated from subject-derived urine samples.

22. The method of claim 20, wherein process (c), in comparison with the normal individual-derived sample, a decrease in the content of the following is diagnosed as atopic dermatitis:
extracellular vesicles derived from one or more bacteria selected from the group consisting of the phylum Cyanobacteria, and the phylum Fusobacteria, which are isolated from subject-derived blood samples, and the phylum Cyanobacteria, which are isolated from subject-derived urine samples,
extracellular vesicles derived from one or more bacteria selected from the group consisting of the class Chloroplast, the class Saprospirae, the class Flavobacteriia, the class Alphaproteobacteria, and the class Fusobacteriia, which are isolated from subject-derived blood samples, and the class Chloroplast, and the class Betaproteobacteria, which are isolated from subject-derived urine samples,
extracellular vesicles derived from one or more bacteria selected from the group consisting of the order Stramenopiles, the order Pseudomonadales, the order Neisseriales, the order Streptophyta, the order Rhizobiales, the order Saprospirales, the order Sphingomonadales, the order Flavobacteriales, the order Caulobacterales, the order Gemellales, the order Pasteurellales, the order Fusobacteriales, the order Rhodobacterales, and the order Bacillales, which are isolated from subject-derived blood samples, and the order MLE1-12, the order Burkholderiales, the order Streptophyta, the order Pseudomonadales, and the order Sphingomonadales, which are isolated from subject-derived urine samples,
extracellular vesicles derived from one or more bacteria selected from the group consisting of the family Exiguobacteraceae, the family Moraxellaceae, the family Bradyrhizobiaceae, the family Rhizobiaceae, the family Flavobacteriaceae, the family Campylobacteraceae, the family Neisseriaceae, the family Pseudomonadaceae, the family Sphingomonadaceae, the family Chitinophagaceae, the family Carnobacteriaceae, the family Caulobacteraceae, the family Weeksellaceae, the family Methylobacteriaceae, the family Gemellaceae, the family Dermabacteraceae, the family Propionibacteriaceae, the family Pasteurellaceae, the family Leptotrichiaceae, the family Oxalobacteraceae, the family Fusobacteriaceae, the family Aerococcaceae, the family Rhodobacteraceae, the family Intrasporangiaceae, the family Paraprevotellaceae, the family Porphyromonadaceae, the family Staphylococcaceae, the family Corynebacteriaceae, the family Tissierellaceae, the family Micrococcaceae, the family Actinomycetaceae, and the family Planococcaceae, which are isolated from subject-derived blood samples, and the family Alcaligenaceae, the family Rhizobiaceae, the family mitochondria, the family Pseudomonadaceae, the family Corynebacteriaceae, the family Comamonadaceae, the family Rhodobacteraceae, and the family Sphingomonadaceae, which are isolated from subject-derived urine samples, or
extracellular vesicles derived from one or more bacteria selected from the group consisting of the genus Exiguobacterium, the genus Acinetobacter, the genus Capnocytophaga, the genus Proteus, the genus Neisseria, the genus Sphingomonas, the genus Pseudomonas, the genus Aggregatibacter, the genus Leptotrichia, the genus Granulicatella, the genus Prevotella, the genus Chryseobacterium, the genus Porphyromonas, the genus Haemophilus, the genus Brachybacterium, the genus Propionibacterium, the genus Eubacterium, the genus Fusobacterium, the genus Enhydrobacter, the genus Paracoccus, the genus Parabacteroides, the genus Staphylococcus, the genus Corynebacterium, the genus Rothia, the genus Actinomyces, the genus Dialister, the genus Faecalibacterium, and the genus Dorea, which are isolated from subject-derived blood samples, and the genus Achromobacter, the genus Agrobacterium, the genus Roseateles, the genus Pseudomonas, the genus Corynebacterium, and the genus Sphingomonas, which are isolated from subject-derived urine samples.
